(19) United States
(12) Patent Application Publication
(10) Pub. No.: US 2018/0205020 A1
(43) Pub. Date: Jul. 19, 2018

(54) EMISSIVE COMPOUNDS AND RELATED DEVICES

(71) Applicant: Massachusetts Institute of Technology, Cambridge, MA (US)

(72) Inventors: Timothy M. Swager, Newton, MA (US); Hiroyuki Tsujimoto, Boston, MA (US); Georgios Markopoulos, Mannheim (DE); Dong-Gwang Ha, Cambridge, MA (US); Marc A. Baldo, Lexington, MA (US)

(73) Assignee: Massachusetts Institute of Technology, Cambridge, MA (US)

(21) Appl. No.: 15/867,004

(22) Filed: Jan. 10, 2018

Related U.S. Application Data
(60) Provisional application No. 62/444,722, filed on Jan. 10, 2017.

(51) Int. Cl. H01L 51/00; C09K 11/06; C07D 417/14; C07D 405/14

(52) U.S. Cl. CPC H01L 51/0067; C09K 11/06; H01L 51/0071; H01L 51/0073; H01L 51/5012; C07D 417/14; C07D 405/14; C09K 2211/1018; H01L 2251/552; H01L 51/0072

(57) ABSTRACT

In one aspect, compositions comprising emissive compounds including polycyclic aromatic groups are provided. In some embodiments, the emissive compounds may include various moieties having desirable physical and electronic properties. In some embodiments, the compositions may be useful for use in, for example, organic light-emitting diodes (OLEDs), chemical sensors, organic photovoltaics, and other devices. An advantageous feature of some embodiments described herein is the ability to tune the electronic properties of the compositions in order to suit a particular application. For example, compositions comprising emissive compounds described herein may exhibit thermally activated delayed fluorescence (TADF) and may be useful as emissive chromophores in e.g., OLED devices. In other cases, the compositions may exhibit high-lying triplet states and may be able to trap various triplet emitters, for use as e.g., host materials for OLED devices. In some cases, the composition may also be readily soluble and processible, and exhibit excellent thermal stability.

### RELATED APPLICATIONS

This application claims priority under 35 U.S.C. § 119(e) to U.S. Provisional Patent Application Ser. No. 62/444,722, filed Jan. 10, 2017, and entitled “Through Space Electronic Interactions For Thermally Activated Delayed Fluorescence,” which is incorporated herein by reference in its entirety for all purposes.

### TECHNICAL FIELD

The present embodiments relate generally to polycyclic aromatic groups, and in some cases, to donor-acceptor polycyclic aromatic groups and associated devices, systems, and compositions.

### BACKGROUND

Efforts to develop materials for high-efficiency organic light emitting diodes (OLEDs) persist, with applications for OLEDs including but not limited to lighting, smartphones, flat panel displays, and emerging flexible displays. In originally developed OLEDs, the internal quantum efficiencies (IQEs) were generally limited by an electron-hole to emitted photon conversion of 25% because non-emissive triplet excitons constituted 75% of the generated excited states. As a result, a maximum of 25% of excitons contributed to the IQE of these OLEDs. Despite the increased efficiency of newer phosphorescent OLED devices based on platinum and iridium, the cost of these rare metals, the difficulty of creating robust blue emitters, and competing triplet-triplet annihilation remain as some of the limitations in these systems. Accordingly, new materials and devices are needed.

### SUMMARY

Optionally substituted polycyclic aromatic groups and associated devices, systems, compositions are provided. The subject matter of this application involves, in some cases, interrelated products, alternative solutions to a particular problem, and/or a plurality of different uses of structures and compositions.

In one aspect, devices are provided. In some embodiments, the device comprises an electrode and an emissive compound in electrical communication with the electrode, the emissive compound comprising a tricyclic aromatic group, optionally substituted, wherein the emissive compound comprises a donor and an acceptor, each bound to the tricyclic aromatic group, wherein the donor comprises an optionally substituted phenothiazine group or an optionally substituted carbazole group, and wherein the acceptor comprises an optionally substituted diphenyltriazine group.

In another aspect, compositions are provided. In some embodiments, the composition comprises an emissive compound comprising a tricyclic aromatic group, optionally substituted, wherein the emissive compound comprises a donor group and an acceptor group, each bound to the tricyclic aromatic group, wherein the donor and acceptor are co-facially aligned, and wherein the highest occupied molecular orbital is localized on the donor and the lowest unoccupied molecular orbital is localized on the acceptor.

In some embodiments, the composition comprises an emissive compound having a structure as in Formula (I):

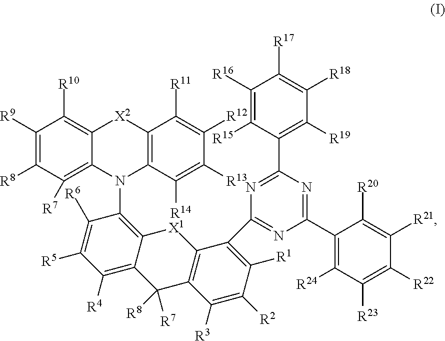

wherein:

X¹ is S, O, or absent,

X² is S, O, N, CH—R²⁵, or absent, and

R¹-R²⁵ are the same or different and are each hydrogen, halo, hydroxyl, amino, alkyl, heteroalkyl, alkenyl, heteroalkenyl, alkynyl, aryl, heteroaryl, heterocyclyl, or carbonyl group, any of which is optionally substituted, and/or any two adjacent groups of R¹-R²⁵ can be joined together to form an optionally substituted ring.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is a schematic diagram of a non-limiting illustrative configuration of a comparative emissive compound comprising a donor and an acceptor, according to certain embodiments;

FIG. 1B is a schematic diagram of a non-limiting illustrative configuration of an emissive compound comprising a donor and an acceptor, according to certain embodiments;

FIG. 1C is a schematic diagram of a non-limiting illustrative configuration of an emissive compound comprising a donor and an acceptor, according to certain embodiments;

FIG. 1D is a schematic diagram of a non-limiting illustrative configuration of an emissive compound comprising a donor and an acceptor, according to certain embodiments;

FIG. 1E is a schematic diagram of a non-limiting illustrative configuration of an emissive compound, XPT, comprising a donor and an acceptor, according to certain embodiments;

FIG. 1F is a schematic diagram of a non-limiting illustrative configuration of an emissive compound, XCT, comprising a donor and an acceptor, according to certain embodiments;

FIG. 1G is a schematic diagram of a non-limiting illustrative configuration of an emissive compound, XtBuCt, comprising a donor and an acceptor, according to certain embodiments;

FIG. 2A is an exemplary diagram of HOMO and LUMO orbital distributions and calculated bandgaps, singlet (S1), triplet (T1) energy levels, and oscillator strengths (f) for emissive compound XPT, according to certain embodiments;

FIG. 2B is an exemplary diagram of HOMO and LUMO orbital distributions and calculated bandgaps, singlet (S1), triplet (T1) energy levels, and oscillator strengths (f) for emissive compound XCT, according to certain embodiments;

FIG. 2C is an exemplary diagram of HOMO and LUMO orbital distributions and calculated bandgaps, singlet (S1), triplet (T1) energy levels, and oscillator strengths (f) for emissive compound XtBuCT, according to certain embodiments;

FIG. 3A shows an exemplary crystal structure for XPT (d=3.423 Å, dashed line), according to certain embodiments;

FIG. 3B shows an exemplary crystal structure for XCT (d=3.375 Å, dashed line), according to certain embodiments;

FIG. 3C shows an exemplary crystal structure for XtBuCT (d=3.299 Å, dashed line), according to certain embodiments;

FIG. 4A is a plot of the PL transient spectrum of XPT in toluene under saturated oxygen and saturated nitrogen at room temperature; concentration of XPT was 1×10-4, λex=336 nm, according to certain embodiments;

FIG. 4B is a plot of PL spectra of XPT in THF/water mixture and the change of normalized PL peak intensity with different water fractions; concentration of XPT was 1×10-5 M, λex=320 nm; inset: PL images of XPT with different water fractions under 365 nm UV light, according to certain embodiments;

FIG. 5A is a schematic diagram of an exemplary organic light emitting device (OLED) structure comprising an emissive compound described herein, according to certain embodiments;

FIG. 5B is a plot of current density-voltage-luminance characteristics of exemplary OLED devices comprising an emissive compound, according to certain embodiments;

FIG. 5C is a plot of external quantum efficiencies (EQEs) of exemplary OLEDs comprising an emissive compound as a function of current density, according to certain embodiments;

FIG. 5D is a plot of electroluminescence (EL) spectrum of exemplary OLED devices comprising an emissive compound at 1 mA/cm2 according to certain embodiments;

FIG. 6 is a schematic illustration of exemplary synthesis schemes for exemplary emissive compounds, according to certain embodiments;

FIG. 7A is a schematic illustration of exemplary synthesis schemes for exemplary emissive compounds, according to certain embodiments;

FIG. 7B is a schematic illustration of exemplary synthesis schemes for exemplary emissive compounds, according to certain embodiments;

FIG. 8 shows photophysical properties of emissive compound XPT in various solutions and thin film, excited at 365 nm, according to certain embodiments;

FIG. 9 shows photophysical properties of emissive compound XCT in various solutions and thin film, excited at 350 nm, according to certain embodiments;

FIG. 10 shows photophysical properties of emissive compound XtBuCT in various solutions and thin film, excited at 320 nm, according to certain embodiments;

FIG. 11A shows a plot of the excitation spectra in toluene (1×10 M) (red solid line) and thin film (blue solid line) of XPT, monitored at 560 nm, 420 nm and 450 nm respectively, according to certain embodiments;

FIG. 11B shows a plot of the excitation spectra in toluene (1×10 M) (red solid line) and thin film (blue solid line) of XCT, monitored at 560 nm, 420 nm and 450 nm respectively, according to certain embodiments;

FIG. 11C shows a plot of the excitation spectra in toluene (1×10 M) (red solid line) and thin film (blue solid line) of XtBuCT, monitored at 560 nm, 420 nm and 450 nm respectively, according to certain embodiments;

FIG. 12A shows a plot of delayed emission spectra in toluene (1×10⁻⁴ M) of XPT, according to certain embodiments;

FIG. 12B shows a plot of delayed emission spectra in toluene (1×10⁻⁴ M) of XCT, according to certain embodiments;

FIG. 12C shows a plot of delayed emission spectra in toluene (1×10⁻⁴ M) of XtBuCT, according to certain embodiments;

FIG. 13A shows a plot of delayed emission spectra of XPT in the thin film state (10 wt % dopant doped in DPEPO) under nitrogen at room temperature, according to certain embodiments;

FIG. 13B shows a plot of delayed emission spectra of XtBuCT in the thin film state (10 wt % dopant doped in DPEPO) under nitrogen at room temperature, according to certain embodiments;

FIG. 14A is a plot of PL spectra of XCT in the THF/water mixtures (1.0×10⁻⁵ M) with different fractions of water (f_{w}), excited at 320 nm, according to certain embodiments;

FIG. 14B is a plot of changes in the PL intensities of XCT with various f_{w}, according to certain embodiments;

FIG. 14C shows PL photographs of a solution in THF and suspension of THF/water mixtures under a 365 nm UV-LED lamp, according to certain embodiments;

FIG. 15A is a plot of PL spectra of XtBuCT in the THF/water mixtures (1.0×10⁻⁵ M) with different fractions of water (f_{w}), excited at 340 nm, according to certain embodiments;

FIG. 15B is a plot of changes in the PL intensities of XtBuCT with various f_{w}, according to certain embodiments;

FIG. 15C shows PL photographs of a solution in THF and suspension of THF/water mixtures under a 365 nm UV-LED lamp, according to certain embodiments;

FIG. 16 is a schematic illustration of an example of molecular packing of XPT, according to certain embodiments;

FIG. 17 is a schematic illustration of an example of molecular packing of XCT, according to certain embodiments;

FIG. 18 is a schematic illustration of an example of molecular packing of XtBuCT, according to certain embodiments;

FIG. 19 is a plot of weight loss versus temperature for XPT, according to certain embodiments;

FIG. 20A is a plot of heat flow (W/g) versus temperature for XPT, according to certain embodiments;

FIG. 20B is a plot of heat flow (W/g) versus temperature for XPT, according to certain embodiments;

FIG. 21 is a plot of weight loss versus temperature for XCT, according to certain embodiments;

FIG. 22A is a plot of heat flow (W/g) versus temperature for XCT, according to certain embodiments;

FIG. 22B is a plot of heat flow (W/g) versus temperature for XCT, according to certain embodiments;

FIG. 23 is a plot of weight loss versus temperature for XtBuCT, according to certain embodiments;

FIG. 24A is a plot of heat flow (W/g) versus temperature for XtBuCT, according to certain embodiments;

FIG. 24B is a plot of heat flow (W/g) versus temperature for XtBuCT, according to certain embodiments;

FIG. 25A shows a plot of oxidation and reduction potentials of XPT, XCT and XtBuCT; oxidation potentials were measured in DCM and reduction potentials were measured in THF solution, according to certain embodiments;

FIG. 25B shows a plot of oxidation and reduction potentials of XPT, XCT and XtBuCT; oxidation potentials were measured in DCM and reduction potentials were measured in THF solution, according to certain embodiments;

FIG. 26 is a plot of absorption spectra of XPT, XCT and XtBuCT in DCM (1×10⁻⁵M), according to certain embodiments;

FIG. 27 is a schematic illustration of the structures of exemplary bridged emissive compounds and exemplary unbridged compounds, according to certain embodiments;

FIG. 28A is a plot of absorption spectra in toluene (1×10⁻⁵ M) of (a) XPT (D-b-A), XP2 (D-b-D), 9PPT (D) and TPTr (A), according to one set of embodiments;

FIG. 28B is a plot showing a comparison between D-b-A and D+A mixture, according to certain embodiments;

FIG. 29 is a plot of PL spectra, excited at 320 nm, of XP2 (D-b-D) in various solvents, according to certain embodiments;

FIG. 30A is a plot of PL spectra, excited at 320 nm, of XPT (D-b-A), XP2(D-b-D) and 9PPT(D) in toluene (1×10⁴ M), according to certain embodiments;

FIG. 30B is a plot of PL spectra, excited at 320 nm, of XPT (D-b-A), XP2(D-b-D) and 9PPT(D) in thin films, according to certain embodiments;

FIG. 31A is a plot of PL spectra, excited at 320 nm, of XPT (D-b-A), 9PPT(D) and the mixture of 9PPT and TPTr (1:1 mol ratio)(D+A) in toluene (1×10⁴ M), according to certain embodiments;

FIG. 31B is a plot of PL spectra, excited at 320 nm, of XPT (D-b-A), 9PPT(D) and the mixture of 9PPT and TPTr (1:1 mol ratio)(D+A) in thin films, according to certain embodiments;

FIG. 32A is a plot of PL spectra, excited at 320 nm, of XCT (D-b-A), 9PCz (D) and the mixture of 9PCz and TPTr (1:1 mol ratio) (D+A) in toluene (1×10⁴ M), according to certain embodiments;

FIG. 32B is a plot of PL spectra, excited at 320 nm, of XCT (D-b-A), 9PCz (D) and the mixture of 9PCz and TPTr (1:1 mol ratio) (D+A) in thin films, according to certain embodiments;

FIG. 33A is a diagram of transition and fluorescence pathway of exemplary bridged structures in solution and in the thin films, according to certain embodiments;

FIG. 33B is a diagram of transition and fluorescence pathway of exemplar unbridged structures in solution, according to certain embodiments;

FIG. 33C is a diagram of transition and fluorescence pathway of exemplar unbridged structures in thin films, according to certain embodiments;

FIG. 34A shows PL photographs of XPT isolated with various ways under 365 nm LED-lamp: (a) switchable PL upon grinding, sublimation and heating and DCM vapor, (b) PL of a single crystal and (c) PL of a spin-coated film before and after heating, according to certain embodiments;

FIG. 34B is a plot of PL spectra of XPT of ground solids, heated solids and DCM vapor fumed solids, according to certain embodiments;

FIG. 34C is a plot of excitation spectra of XPT obtained from various treatments, according to certain embodiments;

FIG. 35A is a plot of the DSC profile of XPT in the first and second cycle as prepared green PL solid, according to certain embodiments;

FIG. 35B is a plot of the DSC profile of XPT in the first and second cycle as a sublimated yellow PL solid, according to certain embodiments;

FIG. 36 shows exemplary powder XRD patterns of XPT, according to certain embodiments; and

FIG. 37 shows reversible switching of emission colors of XPT on a glass substrate under 365 nm LED lamp, according to certain embodiments.

### DETAILED DESCRIPTION

In one aspect, compositions comprising emissive compounds including polycyclic aromatic groups are provided. In some embodiments, the emissive compounds may include various moieties having desirable physical and electronic properties. In some embodiments, the compositions may be useful for use in, for example, organic light-emitting diodes (OLEDs), chemical sensors, organic photovoltaics, and other devices. An advantageous feature of some embodiments described herein is the ability to tune the electronic properties of the compositions in order to suit a particular application. For example, compositions comprising emissive compounds described herein may exhibit thermally activated delayed fluorescence (TADF) and may be useful as emissive chromophores in e.g., OLED devices. In other cases, the compositions may exhibit high-lying triplet states and may be able to trap various triplet emitters, for use as e.g., host materials for OLED devices. In some cases, the composition may also be readily soluble and processible, and exhibit excellent thermal stability.

In some cases, the composition may include an emissive compound comprising a polycyclic aromatic group, optionally substituted, having various functional groups arranged at specific locations within the compound to generate a desired electronic structure or to produce desired electronic properties. For example, arrangement of various electron-withdrawing or electron-deficient groups and/or electron-donating or electron-rich groups within the optionally substituted polycyclic aromatic group may advantageously create relatively low overlap between the highest occupied molecular orbital (HOMO) and the lowest unoccupied molecular orbital (LUMO), relative to other TADF compositions. In some embodiments, and without wishing to be bound by theory, this may result in the optionally substituted polycyclic aromatic group having a relatively small energy gap (ΔE_{ST}) between the lowest singlet and triplet excited states, such that the composition exhibits high photoluminescence efficiency (e.g., high quantum yield) and/or extended lifetimes as compared to traditional TADF compositions.

The phrase “emissive compound” is given its ordinary meaning in the art and generally refers to, for example, chemical compounds which produce electromagnetic radiation (e.g., fluorescence) in response to a stimulus (e.g., an electrical stimulus such as an an applied voltage). As used herein, “emission” may be luminescence emission, in which “luminescence” is defined as an emission of ultraviolet or visible radiation. Specific types of luminescence include fluorescence, in which a time interval between absorption and emission of visible radiation ranges from 10⁻¹² to 10⁻⁷ s, phosphorescence, other types of luminescence such as electroluminescence, and the like. For example, the emission may be “chemiluminescence,” which refers to the emission of radiation due to a chemical reaction, or “electrochemiluminescence,” which refers to emission of radiation due to electrochemical reactions. In some cases, the emission may be fluorescence emission.

In some cases, the emissive compound comprises a donor group and an acceptor group, each bound to an optionally substituted and/or branched polycyclic (e.g., tricyclic) aromatic group, such that the highest occupied orbital is localized on the donor and the lowest unoccupied orbital is localized with the acceptor. In some embodiments, the donor comprises a phenothiazine-based group or a carbazole-based group. In certain embodiments, the acceptor comprises a diphenyltriazine-based group.

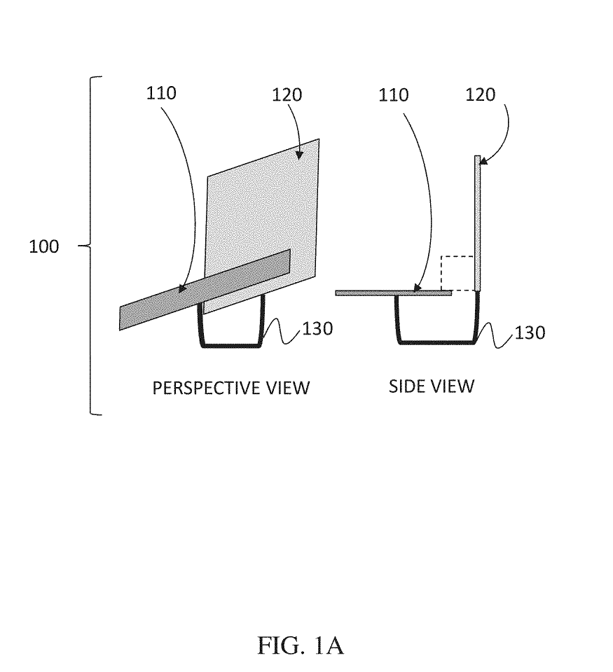

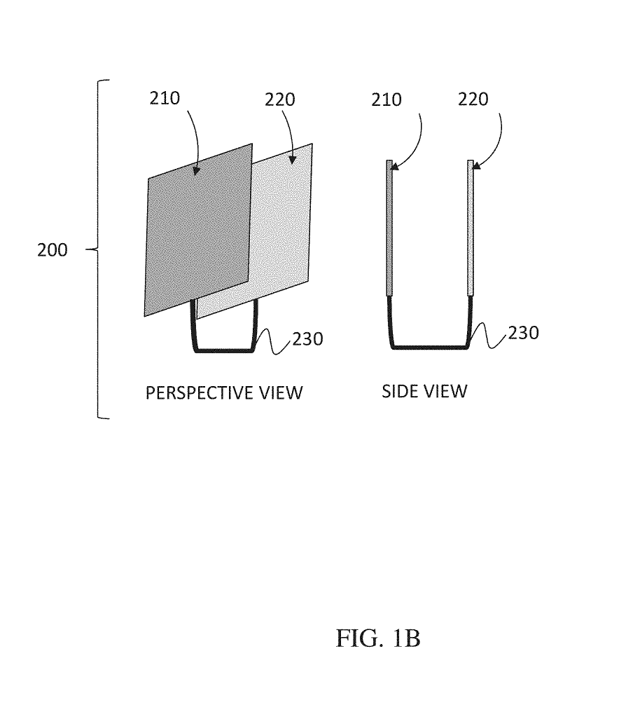

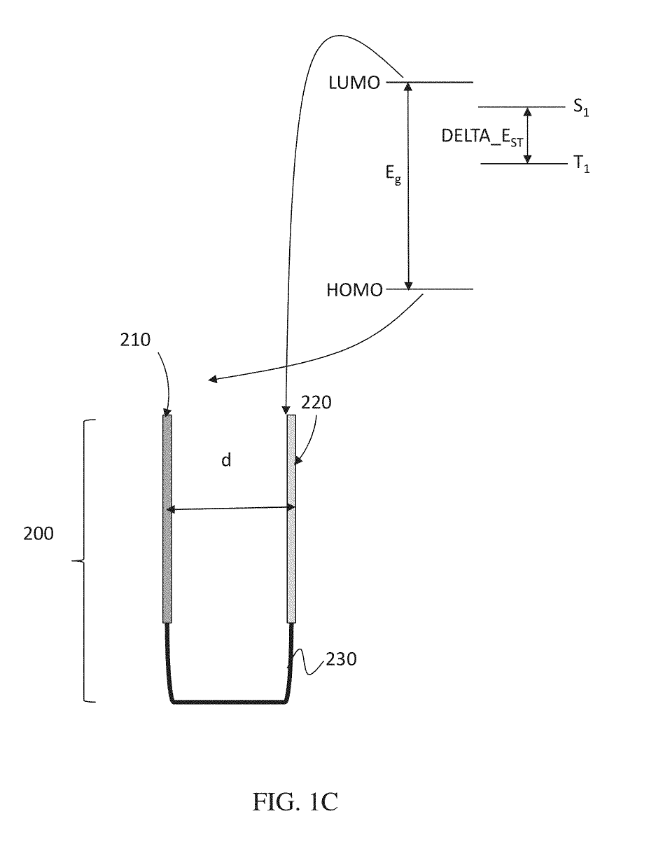

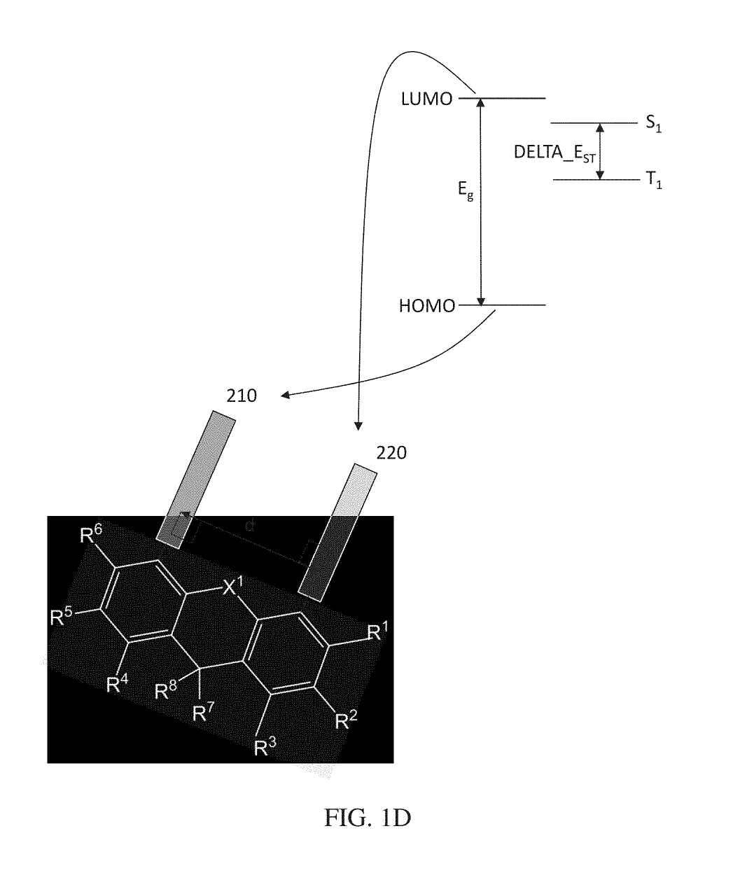

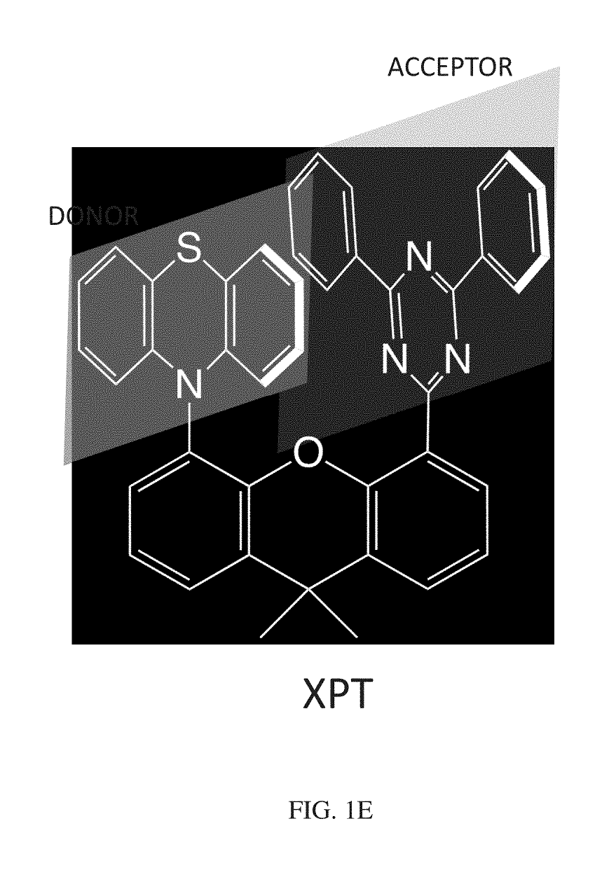

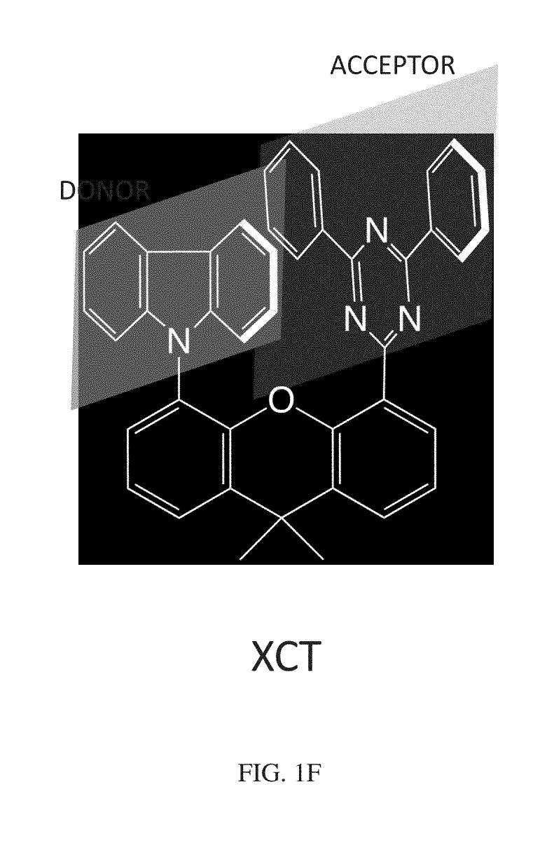

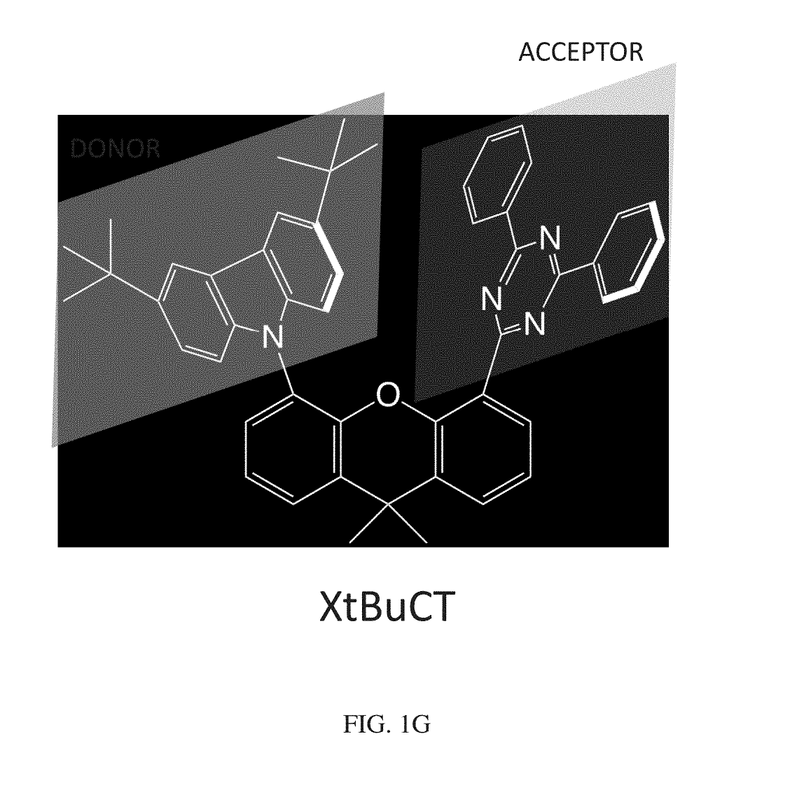

FIG. 1A illustrates an exemplary emissive compound 100 comprising donor group 110 and acceptor group 120 each bound to common compound 130 in perspective and side views, according to some embodiments. FIG. 1B illustrates an exemplary emissive compound 200 comprising donor group 210 and acceptor group 220 each bound to a polycyclic aromatic group 230 (e.g., a tricyclic aromatic group), according to some embodiments. In some cases, the donor group and the acceptor group may be cofacially oriented (e.g., cofacially aligned). For example, as illustrated in FIG. 1B, donor group 210 and acceptor group 220 are cofacially oriented. In some embodiments, a major axial plane of donor group 210 and a major axial plane of acceptor group 220 are parallel to one another. By contrast, as illustrated in FIG. 1A, donor group 110 and acceptor group 120 are not cofacially oriented.

In certain embodiments, the emissive compound may have a U-shaped structure. For example, as illustrated in FIG. 1C, emissive compound 200 (comprising donor group 210 and acceptor group 220 each bound to polycyclic aromatic group 230) has a U-shaped structure.

Any terms as used herein related to shape, orientation, alignment, and/or geometric relationship of or between, for example, one or more structures, compounds, groups and/or combinations thereof and/or any other tangible or intangible elements not listed above amenable to characterization by such terms, unless otherwise defined or indicated, shall be understood to not require absolute conformance to a mathematical definition of such term, but, rather, shall be understood to indicate conformance to the mathematical definition of such term to the extent possible for the subject matter so characterized as would be understood by one skilled in the art most closely related to such subject matter. Examples of such terms related to shape, orientation, alignment, and/or geometric relationship include, but are not limited to terms descriptive of: Examples of such terms related to shape, orientation, and/or geometric relationship include, but are not limited to terms descriptive of: shape—such as, round, square, circular/circle, rectangular/rectangle, triangular/triangle, cylindrical/cylinder, elipitical/elipse, (n)polygonal/(n)polygon, U-shaped, line-shaped, etc.; angular orientation—such as perpendicular, orthogonal, parallel, vertical, horizontal, collinear, etc.; contour and/or trajectory—such as, plane/planar, coplanar, hemispherical, semi-hemispherical, line/linear, hyperbolic, parabolic, flat, curved, straight, arcuate, sinusoidal, tangent/tangential, etc. As one example, a fabricated article that would described herein as being “square” would not require such article to have faces or sides that are perfectly planar or linear and that intersect at angles of exactly 90 degrees (indeed, such an article can only exist as a mathematical abstraction), but rather, the shape of such article should be interpreted as approximating a “square,” as defined mathematically, to an extent typically achievable and achieved for the recited fabrication technique as would be understood by those skilled in the art or as specifically described. As another example, two or more structures that would described herein as being “aligned” (e.g., cofacially aligned) would not require such structures to have faces or sides that are perfectly aligned (indeed, such a structure can only exist as a mathematical abstraction), but rather, the arrangement of such structures should be interpreted as approximating “aligned,” as defined mathematically, to an extent typically achievable and achieved for the recited structure and/or fabrication of such a structure as would be understood by those skilled in the art or as specifically described.

In some embodiments, the emissive compound comprises a tricyclic aromatic group (e.g., a xanthene-based molecule). While much of the description herein relates to tricyclic aromatic groups, lower (e.g., bicyclic) and higher (e.g., quadracyclic, etc.) aromatic groups are also possible. For example, in some embodiments, the emissive compound has a structure as in Formula (I):

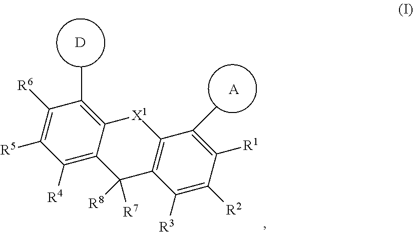

wherein D is a donor group, A is an acceptor group, X¹ is S, O, or absent, and each of R¹-R⁸ are the same or different and are each hydrogen, halo, hydroxyl, amino, alkyl, heteroalkyl, alkenyl, heteroalkenyl, alkynyl, aryl, heteroayrl, heterocyclyl, or carbonyl group, any of which is optionally substituted, and/or any two adjacent groups of R¹-R⁸ can be joined together to form an optionally substituted ring. In an exemplary set of embodiments, R⁷ and R⁸ are each hydrogen or methyl. In some embodiments, the emissive compound is a xanthene-based compound. In certain embodiments, D and A are cofacially aligned. For example, as illustrated in FIG. 1D, donor group 210 and acceptor group 220 are each bound to a tricyclic aromatic group, are cofacially aligned, and separated by an intramolecular distance d.

In some embodiments, the intramolecular distance between the donor group and the acceptor group is less than or equal to 5 angstroms, less than or equal to 4.8 angstroms, less than or equal to 4.5 angstroms, less than or equal to 4.2 angstroms, less than or equal to 4 angstroms, less than or equal to 3.8 angstroms, less than or equal to 3.5 angstroms, less than or equal to 3.3 angstroms, less than or equal to 3 angstroms, or less than or equal to 2.8 angstroms. In certain embodiments, the intramolecular distance between the donor group and the acceptor group is greater than or equal to 2.5 angstroms, greater than or equal to 2.8 angstroms, greater than or equal to 3 angstroms, greater than or equal to 3.3 angstroms, greater than or equal to 3.5 angstroms, greater than or equal to 3.8 angstroms, greater than or equal to 4 angstroms, greater than or equal to 4.2 angstroms, greater than or equal to 4.5 angstroms, or greater than or equal to 4.8 angstroms. Combinations of the above-referenced ranges are also possible (e.g., less than or equal to 5 angstroms and greater than or equal to 2.5 angstroms, less than or equal to 3.8 angstroms and greater than or equal to 4.5 angstroms, less than or equal to 3.3 angstroms and greater than or equal to 3.5 angstroms). Other ranges and combinations are also possible.

In some embodiments, the highest occupied molecular orbital (HOMO) is localized on the donor group. In certain embodiments, the lowest unoccupied molecular orbital (LUMO) is localized on the acceptor group. For example, referring again to FIG. 1C, exemplary emissive compound 200 comprises a HOMO localized on donor group 210 and a LUMO localized on acceptor group 220.

In certain embodiments, the emissive compound has a structure as in Formula (II):

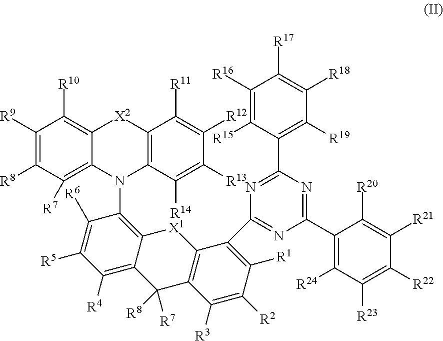

In a particular set of embodiments X¹ is oxygen (e.g., the tricyclic aromatic group comprises a xanthene molecule, optionally substituted). In certain embodiments, X² is S, O, N, CH—R²⁵, or absent. In an exemplary set of embodiments, X² is sulfur. In another exemplary set of embodiments, X² is absent. In yet another exemplary set of embodiments, X² is CH—R²⁵ (e.g., where R²⁵ is hydrogen).

In certain embodiments, R¹-R²⁵ are the same or different and are each hydrogen, halo, hydroxyl, amino, alkyl, heteroalkyl, alkenyl, heteroalkenyl, alkynyl, aryl, heteroayrl, heterocyclyl, or carbonyl group, any of which is optionally substituted, and/or any two adjacent groups of R¹-R²⁵ can be joined together to form an optionally substituted ring. In an exemplary set of embodiments, R⁷ and R⁸ are each hydrogen or methyl, R⁹ and R¹² are each hydrogen or tert-butyl, and R¹-R⁶, R¹⁰-R¹¹, R¹³-R²⁵ are each hydrogen. In another set of exemplary embodiments, R⁷ and R⁸ are each methyl, and R¹-R⁶ and R⁹-R²⁵ are each hydrogen.

In certain embodiments, the donor comprises an optionally substituted phenothiazine group or an optionally substituted carbazole group. In some embodiments, the acceptor comprises an optionally substituted diphenyltriazine group.

In an exemplary set of embodiments, the emissive compound has a structure as in Formula (III):

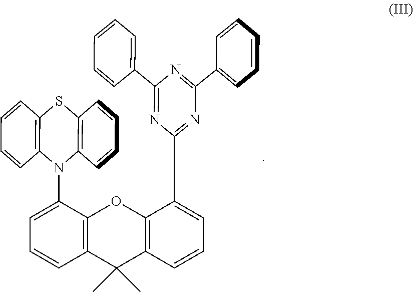

In another exemplary set of embodiments, the emissive compound has a structure as in Formula (IV):

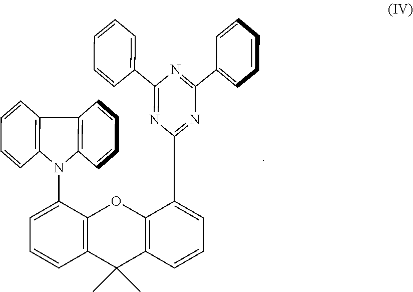

In yet another exemplary set of embodiments, the emissive compound has a structure as in Formula (V):

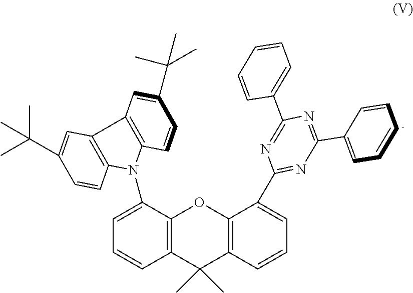

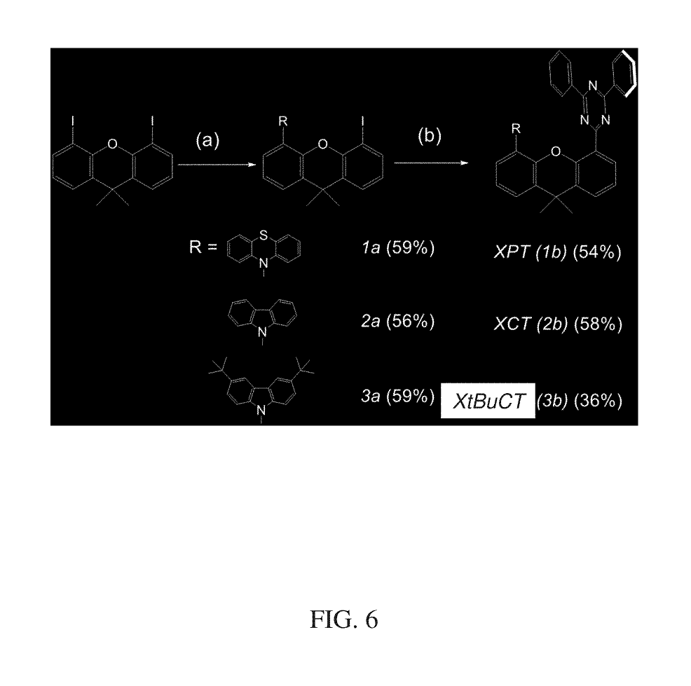

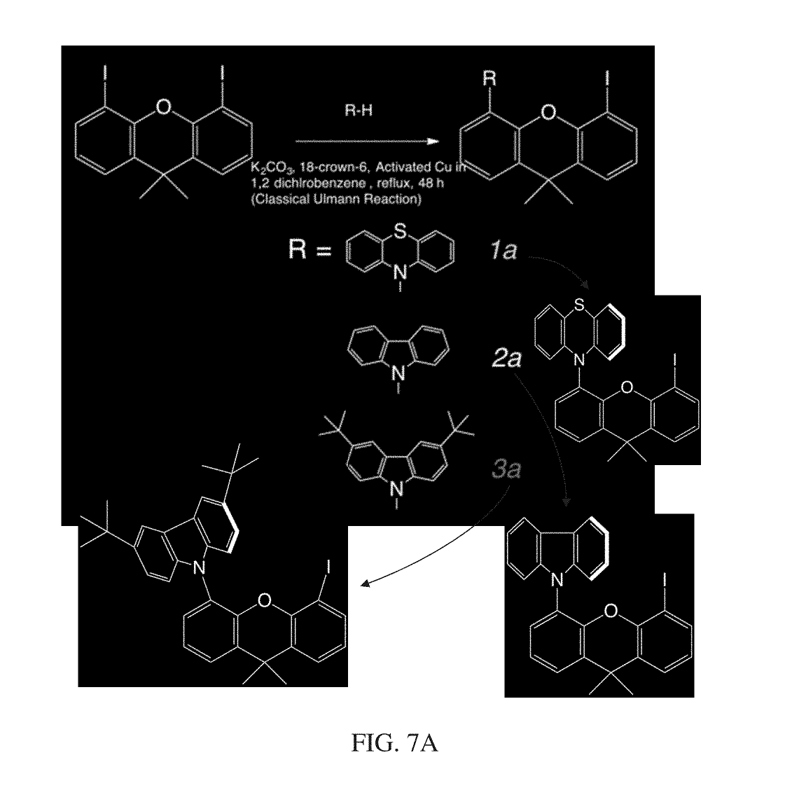

Methods for synthesizing such emissive compounds are described herein, as well as in U.S. Provisional Patent Application Ser. No. 62/444,722, filed Jan. 10, 2017, and entitled “Through Space Electronic Interactions For Thermally Activated Delayed Fluorescence” and Tsujimoto, Hiroyuki, et al. “Thermally Activated Delayed Fluorescence and Aggregation Induced Emission with Through-Space Charge Transfer.” Journal of the American Chemical Society 139.13 (2017): 4894-4900, the content of which are incorporated herein by reference in their entirety for all purposes. FIG. 6, FIG. 7A, and FIG. 7B illustrate the synthesis of exemplary emissive compounds including, according to some embodiments.

The emissive compound may have desirable properties including excited state lifetimes, efficiency of emission, and/or wavelength of emission.

In some embodiments, an thermally activated delayed fluorescence (TADF) and/or excited state lifetime of the emissive compound is greater than or equal to 0.1 microsecond, greater than or equal to 0.5 microseconds, greater than or equal to 1 microseconds, greater than or equal to 3 microseconds, greater than or equal to 5 microseconds, greater than or equal to 10 microseconds, or greater than or equal to 20 microseconds In certain embodiments, the emissive compound has a TADF and/or excited state lifetime of less than or equal to 50 microseconds, less than or equal to 20 microseconds, less than or equal to 10 microseconds, less than or equal to 5 microseconds, less than or equal to 3 microseconds, less than or equal to 2 microseconds, less than or equal to 1 microsecond, or less than or equal to 0.5 microseconds. Combinations of the above-referenced ranges are also possible (e.g., between 0.1 microsecond and 50 microseconds, between 1 microsecond and 5 microseconds). Other ranges are also possible. Those skilled in the art would be capable of selecting methods for determining singlet emission lifetimes including, for example, by time resolved detection of the emission.

The emissive compounds described herein may be configured such that they emit a particular wavelength of electromagnetic radiation (i.e. light). The wavelength of an emission refers to the wavelength at which the peak maximum of the emission occurs in an emission spectrum. The emission may be a particular peak having the largest intensity in an emission spectrum (e.g. a fluorescence spectrum), or, alternatively, the emission may be a peak in an emission spectrum that has at least a defined maximum, but has a smaller intensity relative to other peaks in the emission spectrum.

In some embodiments, the emissive compound described herein has particular peak emission wavelength (e.g., a peak emission wavelength in solution or in a host matrix). In some embodiments, the peak emission wavelength of light emitted by emissive compound (or a device including the emissive compound) is between 300 nm and 700 nm. For example, in some embodiments, the peak emission wavelength of light emitted by the emissive compound may be greater than or equal to 370 nm, greater than or equal to 400 nm, greater than or equal to 500 nm, or greater than or equal to 600 nm. In certain embodiments, the peak emission wavelength of light emitted by the emissive compound may be less than 700 nm, less than 600 nm, less than 500 nm, or less than 400 nm. Combinations of the above-referenced ranges are also possible (e.g., an average wavelength between 370 nm and 700 nm, between 400 nm and 600 nm, between 400 nm and 500 nm).

In a particular set of embodiments, the emissive compound may have a peak emission wavelength of at least 400 nm, at least 420 nm, at least 440 nm, at least 460 nm, at least 480 nm, at least 500 nm, at least 520 nm, at least 540 nm, or at least 560 nm. In certain embodiments, the emissive compound may have a peak emission wavelength of less than or equal to 580 nm, less than or equal to 560 nm, less than or equal to 540 nm, less than or equal to 520 nm, less than or equal to 500 nm, less than or equal to 480 nm, less than or equal to 460 nm, less than or equal to 440 nm, or less than or equal to 420 nm. Combinations of the above-referenced ranges are also possible (e.g., between 400 nm and 580 nm, between 400 nm and 460 nm). Devices comprising the emissive compounds described herein may have an average electroluminescence wavelength in the same range as the peak emission wavelength of the emissive compound (e.g., between 400 nm and 580 nm). For example, in some embodiments, applying an electric potential to a device comprising a layer comprising the emissive compound may generate light having average wavelength ranging between 400 nm and 580 nm. Devices and methods for generating light are described in more detail, below.

In a particular set of embodiments, the wavelength of emission of the emissive compound (or a device comprising the emissive compound) is less than or equal to 500 nm. In some cases, the emission of the emissive compound (or the device comprising the emissive compound) may be characterized as giving blue light. In some embodiments, the missive compound emits blue light when excited (e.g., when an electric potential is applied) in a solid state.

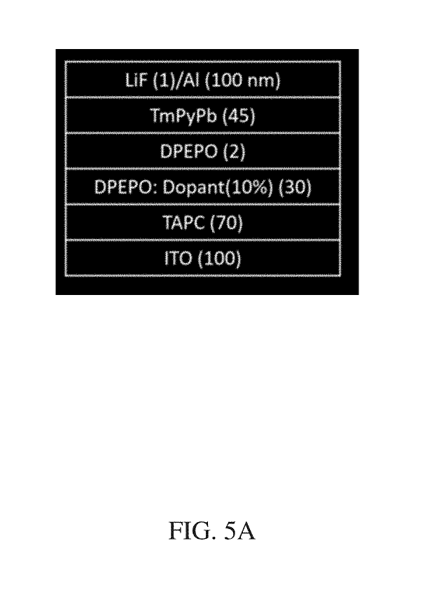

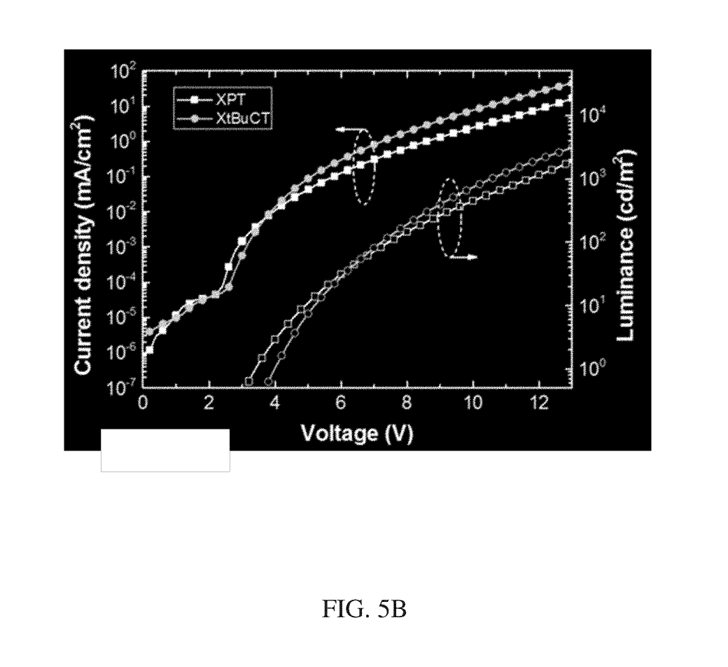

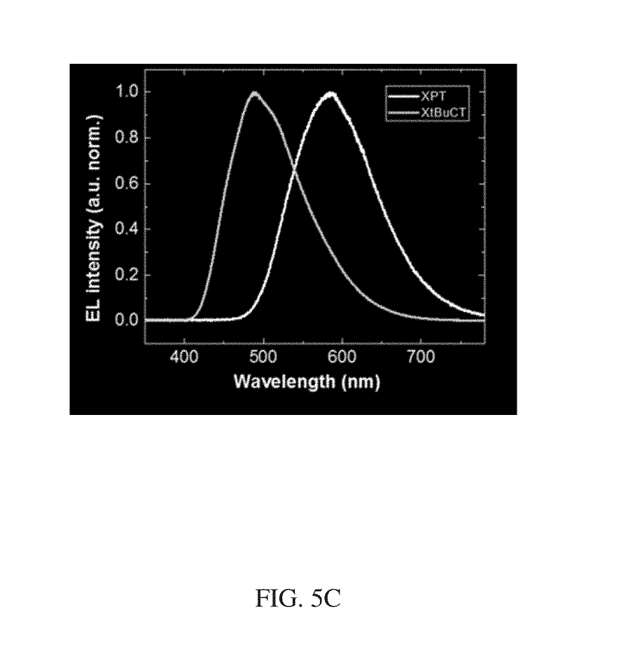

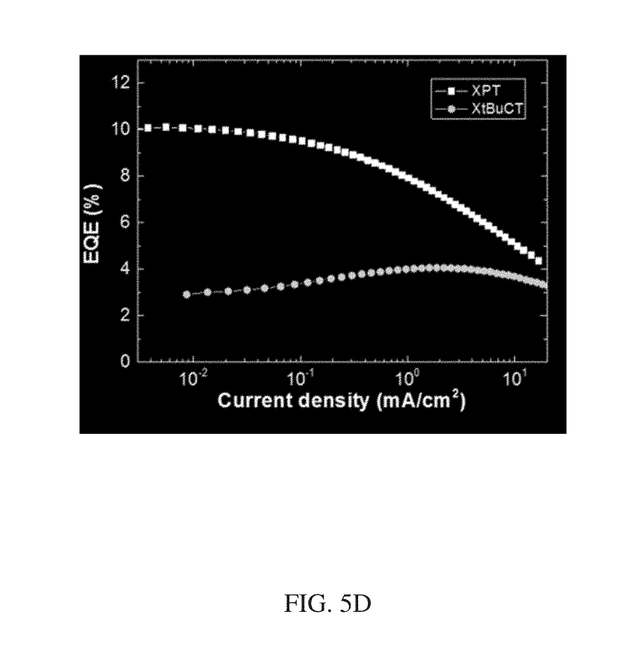

Devices incorporating the emissive compound disclosed herein are also provided. For example, the emissive compound may be useful as a dopant and/or chromophore in a luminescence-based device such as an OLED. An exemplary device is shown schematically in FIG. 5A.

Such devices may advantageously exhibit TADF with increased quantum yields and/or quantum efficiencies. In some cases, the emissive compound and structures disclosed herein can be used to create OLED devices that exhibit quantum yield of greater than, about 35%, about 40%, about 45%, about 50%, about 55%, about 60%, or greater. In some cases, devices incorporating the emissive compound and structures disclosed herein may exhibit an internal quantum yields in solution and/or the solid state of about 70%, about 75%, about 80%, about 85%, about 90%, or greater. In some embodiments, the quantum yield of the emissive compound (or device comprising the emissive compound) is determined in the solid state.

In some embodiments, a maximum electroluminescence external quantum efficiency of the device comprising the emissive compound may be greater than or equal to 4%, greater than or equal to 5%, greater than or equal to 6%, greater than or equal to 7%, greater than or equal to 8%, greater than or equal to 9%, or greater than or equal to 10%. In certain embodiments, the maximum electroluminescence external quantum efficiency of the device comprising the emissive compound is less than or equal to 11%, less than or equal to 10%, less than or equal to 9%, less than or equal to 8%, less than or equal to 7%, less than or equal to 6%, or less than or equal to 5%. Combinations of the above-referenced ranges are also possible (e.g., greater than or equal to 4% and less than or equal to 11%). Other ranges are also possible. In some cases, the efficiency of an emission of the emissive compound is increased in the solid state relative to solution.

Some embodiments may provide the emissive compound combined with, dispersed within, covalently bonded to, coated with, formed on, or otherwise associated with, one or more materials (e.g., small molecules, polymers, metals, metal complexes, etc.) to form a film or layer in solid state. For example, the emissive compound may be combined with an electroactive material to form a film. In some cases, the emissive compound may be combined with a hole-transport polymer. In some cases, the emissive compound may be combined with an electron-transport polymer. In some cases, the emissive compound may be combined with a hole-transport polymer and an electron-transport polymer. In some cases, the emissive compound may be combined with a copolymer comprising both hole-transport portions and electron-transport portions. In such embodiments, electrons and/or holes formed within the solid film or layer may interact with the emissive compound.

Compounds and compositions described herein may be incorporated into various light-sensitive or light-activated devices, such as OLEDs, emissive sensors, or photovoltaic devices. In some embodiments, the composition may be useful in facilitating charge transfer or energy transfer within a device and/or as a hole-transport material. The device may be, for example, an organic light-emitting diode (OLED), an organic integrated circuit (O-IC), an organic field-effect transistor (O-FET), an organic thin-film transistor (O-TFT), an organic light-emitting transistor (O-LET), an organic solar cell (O-SC), an organic optical detector, an organic photoreceptor, an organic field-quench device (O-FQD), a light-emitting electrochemical cell (LEC), an emissive chemosensor, or an organic laser diode (O-laser).

In some embodiments, the device may be an OLED including a composition as described herein. An OLED device typically includes a multilayer stack including a substrate, one or more electrodes (e.g., a cathode, an anode) and one or more layers including a material capable of emitting light, i.e., an emission layer or light-emitting layer. For example, the OLED device may include an emission layer containing a host material and a guest material, and within which excitons are produced. The layer may be positioned between and in electrical communication with an anode and a cathode. Other additional layers within an OLED may include electron-transporting layers, electron-injecting layer, hole-injecting layers, hole-transporting layers, exciton-blocking layers, spacer layers, connecting layers, hole-blocking layers, and the like. In some cases, the OLED may be a fluorescence-based OLED (e.g., TADF-based OLED). In some cases, the OLED may be a phosphorescence-based OLED. OLED devices, and methods for forming OLEDs, will be known to those of ordinary skill in the art. An illustrative embodiment of an OLED device is shown in FIG. 5A.

In a typical OLED, holes and electrons injected into the device can recombine to form excitons, including, in the case of a phosphorescence-based OLED, both singlet and triplet excitons. In some cases, compositions described herein may facilitate the generation and/or retention of, a greater number of triplet excitons relative to singlet excitons. This may be desirable in certain OLEDS, to transform triplet excitons into singlet excitons to create more efficient emission such that a 100% internal quantum efficiency is theoretically possible.

In some cases, compositions described herein may serve as a chromophore within an OLED device.

In some embodiments, it may be desirable to include a hole-blocking layer within the OLED device to help confine the excitons and recombination events to the emission layer. Some examples of hole-blocking materials are described in International Publications WO 00/70655A2, WO 01/41512, and WO 01/93642. Those of ordinary skill in the art would be capable of selecting hole-transport materials, or mixtures thereof, suitable for use in embodiments described herein.

Those of ordinary skill in the art would be capable of selecting appropriate cathode materials for use in embodiments described herein. In some cases, the cathode material may be a hole conducting material. In some cases, the anode material may be substantially transparent. The anode material may be selected to promote electron injection at low voltage, and have effective stability. Examples of cathode materials are described in U.S. Pat. Nos. 4,885,211; 5,059,861; 5,059,862; 5,247,190; 5,703,436; 5,608,287; 5,837,391; 5,677,572; 5,776,622; 5,776,623; 5,714,838; 5,969,474; 5,739,545; 5,981,306; 6,137,223; 6,140,763; 6,172,459; 6,278,236; and 6,284,393; and European Patent No. 1076368. Cathode materials may be formed within the device using known methods, including thermal evaporation, electron beam evaporation, ion sputtering, or chemical vapor deposition. In some cases, the cathode may be patterned using known photolithographic processes.

In some embodiments, the anode may be selected to be substantially transparent opaque, or reflective. In one set of embodiments, the anode may be substantially transparent to the emission generated by the emission later. Examples of transparent anode materials include metal oxides such as indium-tin oxide (ITO), indium-zinc oxide (IZO), tin oxide, aluminum- or indium-doped zinc oxide, magnesium-indium oxide, and nickel-tungsten oxide, metal nitrides such as gallium nitride, metal selenides such as zinc selenide, and metal sulfides such as zinc sulfide. The anode may be formed within the devices using known techniques such as evaporation, sputtering, chemical vapor deposition, or electrochemical techniques. In some cases, the anode may be patterned using known photolithographic processes. In some cases the device can have layers that shift the emission color to create devices that have desirable color in their emission.

The substrate can be any material capable of supporting the device components as described herein. Preferably, the substrate material has a thermal coefficient of expansion similar to those of the other components of the device to promote adhesion and prevent separation of the layers at various temperatures. In some instances, materials with dissimilar thermal expansion coefficients may expand and contract at different rates and amounts with changes in temperature, which can cause stress and delamination of the layers. The substrate can either be light transmissive or opaque, depending on the intended direction of light emission. Examples of appropriate substrate materials may include glass, plastic, semiconductor materials such as silicon, ceramics, and circuit board materials. In some instances, it may be advantageous to have the materials be non-crystalline such that grain boundaries between different crystalline domains do not develop during device formation or during operation of the device.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The alkyl groups may be optionally substituted, as described more fully below. Examples of alkyl groups include, but are not limited to, methyl, ethyl, propyl, isopropyl, butyl, isobutyl, tert-butyl, 2-ethylhexyl, cyclopropyl, cyclobutyl, cyclopentyl, cyclohexyl, and the like. “Heteroalkyl” groups are alkyl groups wherein at least one atom is a heteroatom (e.g., oxygen, sulfur, nitrogen, phosphorus, etc.), with the remainder of the atoms being carbon atoms. Examples of heteroalkyl groups include, but are not limited to, alkoxy, poly(ethylene glycol)-, alkyl-substituted amino, tetrahydrofuranyl, piperidinyl, morpholinyl, etc. “Fluoroalkyl” groups are alkyl groups wherein at least one hydrogen is replaced with a fluoro group. In some cases, all hydrogen groups of an alkyl group are replaced with fluoro groups to form a fluoroalkyl group (e.g., CF₃).

The term “alkoxy” refers to —O-alkyl. A “fluoroalkoxy” group refers to —O— fluoroalkyl.

The terms “alkenyl” and “alkynyl” refer to unsaturated aliphatic groups analogous to the alkyl groups described above, but containing at least one double or triple bond respectively. The “heteroalkenyl” and “heteroalkynyl” refer to alkenyl and alkynyl groups as described herein in which one or more atoms is a heteroatom (e.g., oxygen, nitrogen, sulfur, and the like).

The term “aryl” refers to an aromatic carbocyclic group having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl), all optionally substituted. “Fluoroaryl” groups are aryl groups that are substituted with at least one fluoro group.

The terms “amine” and “amino” refer to both unsubstituted and substituted amines, e.g., a moiety that can be represented by the general formula: N(R′)(R″)(R′″) wherein R′, R″, and R′″ each independently represent a group permitted by the rules of valence.

The terms “acyl,” “carboxyl group,” or “carbonyl group” are recognized in the art and can include such moieties as can be represented by the general formula:

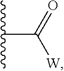

wherein W is H, OH, O-alkyl, O-alkenyl, or a salt thereof. Where W is O-alkyl, the formula represents an “ester.” Where W is OH, the formula represents a “carboxylic acid.” In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, aryl, or another carbon-containing substituent, the above formula represents a “ketone” group. Where W is hydrogen, the above formula represents an “aldehyde” group.

The terms “electron-withdrawing group,” “electron-deficient group,” and “electron-poor group” are recognized in the art and as used herein refer to a functionality which draws electrons to itself more than a hydrogen atom would at the same position. Examples of electron-withdrawing groups include carbonyl groups (e.g., ketone, esters, aldehydes), sulfonyl, fluoro, trifluoromethyl, nitro, cyano, and the like.

The terms “electron-donating group” and “electron-rich group” as used herein refer to a functionality which draws electrons to itself less than a hydrogen atom would at the same position. Exemplary electron-donating groups include amino, hydroxy, alkoxy, acylamino, acyloxy, alkyl, halides, and the like.

As used herein, the term “heterocycle” or “heterocyclyl” refers to a monocyclic or polycyclic heterocyclic ring that is either a saturated ring or an unsaturated non-aromatic ring. Typically, the heterocycle may include 3-membered to 14-membered rings. In some cases, 3-membered heterocycle can contain up to 3 heteroatoms, and a 4- to 14-membered heterocycle can contain from 1 to about 8 heteroatoms. Each heteroatom can be independently selected from nitrogen, which can be quaternized; oxygen; and sulfur, including sulfoxide and sulfone. The heterocycle may be attached via any heteroatom ring atom or carbon ring atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, the hydrogen on a nitrogen may be substituted with a tert-butoxycarbonyl group. Furthermore, the heterocyclyl may be optionally substituted with one or more substituents (including without limitation a halogen atom, an alkyl radical, or aryl radical). Only stable isomers of such substituted heterocyclic groups are contemplated in this definition.

As used herein, the term “heteroaromatic” or “heteroaryl” means a monocyclic or polycyclic heteroaromatic ring (or radical thereof) comprising carbon atom ring members and one or more heteroatom ring members (such as, for example, oxygen, sulfur or nitrogen). Typically, the heteroaromatic ring has from 5 to about 8 ring members in which at least 1 ring member is a heteroatom selected from oxygen, sulfur, and nitrogen. In another embodiment, the heteroaromatic ring is a 5 or 6 membered ring and may contain from 1 to about 4 heteroatoms. In another embodiment, the heteroaromatic ring system has a 7 to 8 ring members and may contain from 1 to about 6 heteroatoms. Representative heteroaryls include pyridyl, furyl, thienyl, pyrrolyl, oxazolyl, imidazolyl, indolizinyl, thiazolyl, isoxazolyl, pyrazolyl, isothiazolyl, pyridazinyl, pyrimidinyl, pyrazinyl, triazinyl, triazolyl, pyridinyl, thiadiazolyl, pyrazinyl, quinolyl, isoquinolyl, indazolyl, benzoxazolyl, benzofuryl, benzothiazolyl, indolizinyl, imidazopyridinyl, isothiazolyl, tetrazolyl, benzimidazolyl, benzoxazolyl, benzothiazolyl, benzothiadiazolyl, benzoxadiazolyl, carbazolyl, indolyl, tetrahydroindolyl, azaindolyl, imidazopyridyl, qunizaolinyl, purinyl, pyrrolo[2,3]pyrimidyl, pyrazolo[3,4]pyrimidyl, benzo(b)thienyl, and the like. These heteroaryl groups may be optionally substituted with one or more substituents.

Suitable substituents for various groups described herein, e.g., alkyl, alkoxy, alkyl sulfanyl, alkylamino, dialkylamino, alkylene, alkenyl, alkynyl, cycloalkyl, cycloalkenyl, heterocyclyl, aryl, aralkyl, heteroaryl, and heteroarylalkyl groups, include any substituent that will form a stable compound. Examples of substituents include alkyl, alkoxy, alkyl sulfanyl, alkylamino, dialkylamino, alkenyl, alkynyl, cycloalkyl, an cycloalkenyl, an heterocyclyl, an aryl, an heteroaryl, an aralkyl, an heteroaralkyl, a haloalkyl, —C(O)NR^{a}R^{b}, —NR^{c}C(O)R^{d}, halo, —OR^{c}, cyano, nitro, haloalkoxy, —C(O)R^{c}, —NR^{a}R^{b}, —SR^{c}, —C(O)OR^{c}, —OC(O)R^{c}, —NR^{c}C(O)NR^{a}R^{b}, OC(O)NR^{a}R^{b}, NR^{c}C(O)OR^{d}, S(O)ₚR^{c}, or —S(O)ₚNR^{a}R^{b}, wherein R^{a} and R^{b}, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl; or R^{a} and R^{b} taken together with the nitrogen to which they are attached form optionally substituted heterocyclyl or optionally substituted heteroaryl; and R^{c} and R^{d} for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocyclyl, an optionally substituted aryl, an optionally substituted heteroaryl, an optionally substituted aralkyl, or an optionally substituted heteroaralkyl. In addition, alkyl, cycloalkyl, alkylene, heterocyclyl, and any saturated portion of a alkenyl, cycloalkenyl, alkynyl, aralkyl, or heteroaralkyl group, may also be substituted with ═O, ═S, or ═NR^{c}.

Choices and combinations of substituents and variables envisioned by embodiments described herein are only those that result in the formation of stable compounds. The term “stable” refers to compounds which possess stability sufficient to allow manufacture and which maintains the integrity of the compound for a sufficient period of time to be useful for the purposes detailed herein (e.g., incorporation within devices such as OLEDs). Typically, such compounds are stable at a temperature of 40° C. or less, in the absence of excessive moisture, for at least one week. Such choices and combinations will be apparent to those of ordinary skill in the art and may be determined without undue experimentation. Unless indicated otherwise, the compounds described herein containing reactive functional groups (such as, without limitation, carboxy, hydroxy, and amino moieties) also include protected derivatives thereof. “Protected derivatives” are those compounds in which a reactive site or sites are blocked with one or more protecting groups. Suitable protecting groups for carboxy moieties include benzyl, tert-butyl, and the like. Suitable protecting groups for amino and amido groups include acetyl, tert-butoxycarbonyl, benzyloxycarbonyl, and the like. Suitable protecting groups for hydroxy include benzyl and the like. Other suitable protecting groups are well known to those of ordinary skill in the art and include those found in T. W. Greene, PROTECTING GROUPS IN ORGANIC SYNTHESIS, John Wiley & Sons, Inc. 1981, the entire teachings of which are incorporated herein by reference for all purposes.

The following examples are intended to illustrate certain embodiments of the present disclosure, but do not exemplify the full scope of the disclosure.

### EXAMPLES

Emissive molecules, each comprising a donor and an acceptor bridged by 9,9 dimethylxanthene, were studied. The structures of the molecules positioned the donor and the acceptor with co-facial alignment at a distance of greater than or equal to about 3.3 Angstroms and less than or equal to about 3.5 Angstroms wherein efficient spatial charge transfer could occur. The quantum yields were enhanced by excluding molecular oxygen and thermally activated delayed fluorescence (TADF) with lifetimes on the order of microseconds was observed. Higher quantum yields were observed for the molecules in the solid state than in solution. Crystal structures revealed pi-pi intramolecular interactions between a donor and an acceptor. However, the dominant intermolecular interactions were C—H . . . pi, which may, in some cases, restrict the molecular dynamics to create aggregation-induced enhanced emission (AIE). Organic light emitting devices using the emissive compounds as dopants displayed electroluminescence external quantum efficiencies (EQE) as high as 10%.

Thermally activated delayed fluorescence (TADF) was used in this example, as an alternative strategy to that of phosphorescent OLED devices. TADF utilizes the up-conversion from triplet excitons to singlet states by reverse intersystem crossing (RISC). With low non-radiative rates and efficient singlet emission, in principle the TADF approach can result in devices with near 100% internal quantum efficiency (IQE). The majority of other TADF molecular designs have utilized conformational effects to twist donor and/or acceptor pi systems from co-planarity to minimize overlap of the highest occupied molecular orbital (HOMO) and lowest unoccupied molecular orbital (LUMO) states (see e.g., FIG. 1A, 100). A low overlap limits the exchange energy gained in the triplet state and results in a small energy difference between singlet and triplet states (DELTA_E_{ST}). However, many twisted TADF materials suffer from reduced quantum yields resulting from pi-pi intermolecular interactions in the solid state.

In this example, a family of donor-acceptor (D-A) xanthene molecules were developed as new high efficiency TADF emitters designed to have intramolecular through space D-A pi-pi interactions. The molecular designs focused on a non-planar molecular scaffold that co-facially organized a donor and an acceptor at a well-controlled distance (see e.g., FIG. 1B, 110). These U-shaped space-through architectures shown in this example (see e.g., FIG. 1B, 110) are non-limiting examples of the donor-acceptor exciplex configuration (see e.g., FIG. 1B, 120). This structure allowed small DELTA_E_{ST} and the further restriction in the solid (aggregated) state provided for enhanced emission that was considered to be aggregation induced delayed fluorescence (AIDF). Molecular structures showing the location of the donor and of the acceptor are depicted for the specific molecules developed in this example, abbreviated herein as XPT (FIG. 1E), XCT (FIG. 1F), and XtBuCT (FIG. 1G).

### Calculations

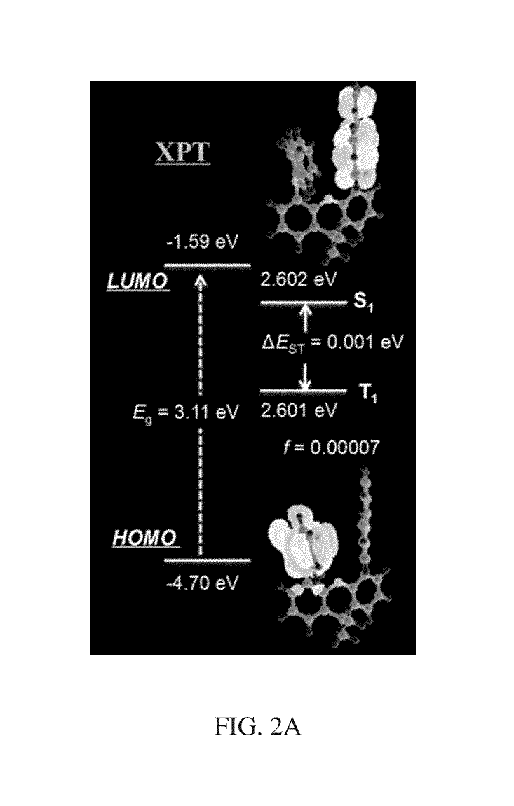

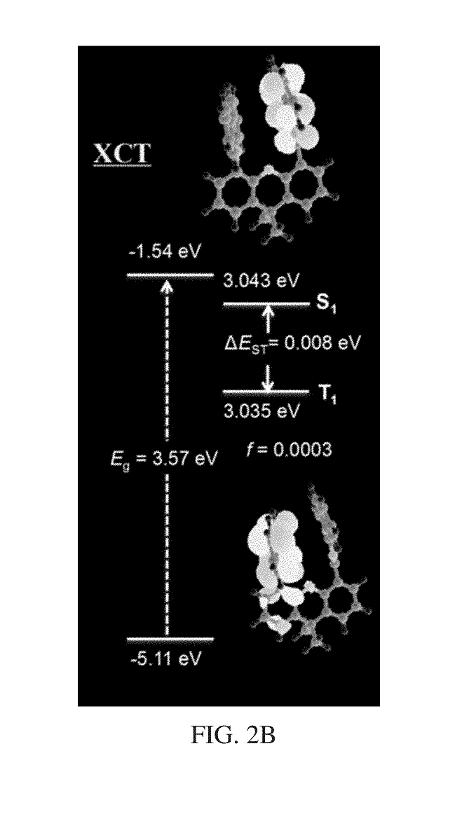

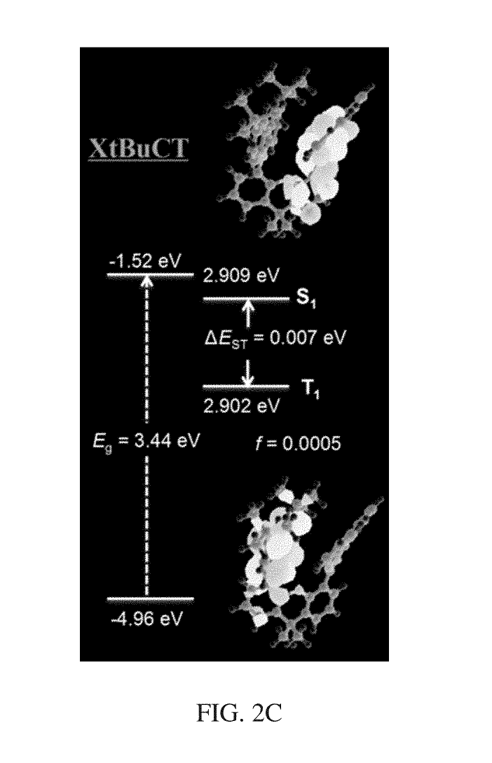

In the designed molecules, a donor and an acceptor were bridged through the 4 and 5 positions of a 9,9-dimethylxanthene scaffold, which generated an inter-chromophore spacing (assuming the pi-systems to be aligned in parallel planes) of 4.7 Angstroms. These designs were guided by time-dependent density functional theory (TD-DFT) based on the B3LYP functional and a 6-31G* basis. Three different donors were examined with different electron donating characters to control the photoluminescence (PL) color (see e.g., FIG. 1B, FIG. 1C, FIG. 1D). The geometry optimizations in the gas phase revealed a U-shape and co-facial intramolecular alignment of a donor and an acceptor with a distance of greater than or equal to about 3.8 Angstroms and less than or equal to about 4.5 Angstroms. The HOMO and LUMO orbital distributions, DELTA_E_{ST}, and oscillator strengths of these molecules are shown in FIG. 2A, FIG. 2B, FIG. 2C, and summarized in Table 1.

TABLE 1

| | HOMO | LUMO | E_{g}/eV | S₁eV | T₁/eV | ΔE_{ST}/eV | f |
|---|---|---|---|---|---|---|---|
| XPT | −4.7030 | −1.587 | 3.1156 | 2.602 | 2.601 | 0.001 | 0.00007 |
| XCT | −5.1135 | −1.537 | 3.5761 | 3.043 | 3.035 | 0.008 | 0.0003 |
| XtBuCt | −4.9578 | −1.52 | 3.4383 | 2.909 | 2.902 | 0.007 | 0.00053 |

FIG. 2A, FIG. 2B, and FIG. 2C show the HOMO and LUMO orbital distributions and calculated bandgaps, singlet (S1) energy levels, triplet (T1) energy levels, and oscillator strengths (f) for XPT, XCT and XtBuCT respectively based on TD-DFT at the B3LYP functional and 6-31G* basis set. The HOMO orbitals were localized over donor groups and the LUMO orbitals were primarily distributed over 2,4-diphenyl-1,3,5-triazine moiety. For XtBuCT, the LUMO orbitals had limited delocalization onto the phenyl ring of the xanthene backbone. The low computed HOMO/LUMO orbital overlaps gave rise to small DELTA_E_{ST} values (greater than or equal to about 1 meV and less than or equal to about 8 meV), suggesting the prospect for rapid equilibration of the lowest triplet (T₁) and singlet (S₁) states (see e.g., Table 1). The low overlap led to small oscillator strengths (f was greater than or equal to about 0.00007 and less than or equal to about 0.0005; see e.g., Table 1). However, with suppression of competitive vibronic couplings facilitating non-radiative decay, highly efficient luminescence could be achieved.

### Synthesis and Characterization

The 9,9-dimethylxanthene-based donor/acceptor chromophores were synthesized as shown in FIG. 6 and the details of all procedures and characterization are given in the supporting information. The donor groups (phenothiazine for XPT, carbazole for XCT and 3,6-di-tert-butylcarbazole for XtBuCT) were installed on the 4-carbon position of xanthene via Ullmann reactions using an activated copper bronze as the catalyst to generate mono-substituted intermediates (see e.g., FIG. 6 and FIG. 7A: 1a, 2a and 3a). The acceptor unit was installed via Negishi cross-coupling reactions using Pd(0) with CPhos and (4,6-diphenyl-1,3,5-triazin-2-yl)zinc(II) bromide, which was prepared in situ via an iodide-magnesium exchange and followed by transmetallation (see e.g., FIG. 6 and FIG. 7B: 1b, 2b and 3b). The synthesis conditions for FIG. 6 (a) were: R-H, K₂CO₃, 18-Crown-6, Activated copper bronze, 1,2-dichlorobenzene, reflux, 48 hours. The synthesis conditions for FIG. 6 (b) were: (i) 2-iodo-4,6-diphenyl-1,3,5-triazine, n-BuMgCl, −78 degrees Celsius, THF, 10 min (ii) ZnBr₂LiCl, −78 degrees Celsius, THF, 15 min, (iii) Pd₂dba₃ (5 mol %), CPhos (15 mol %), THF, reflux, 16 hours.

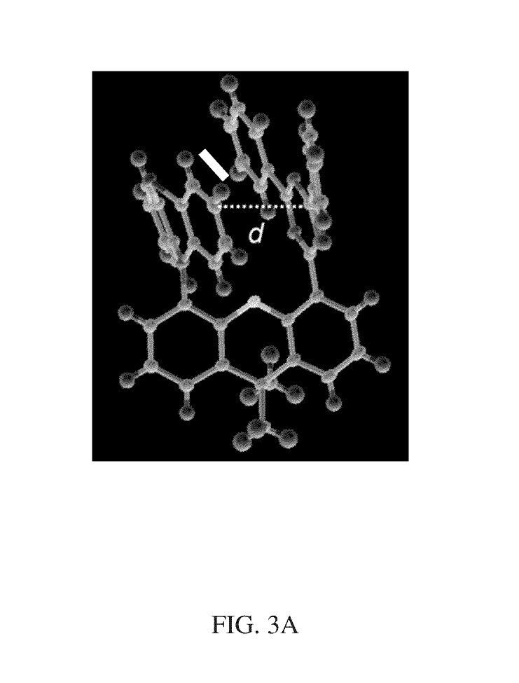

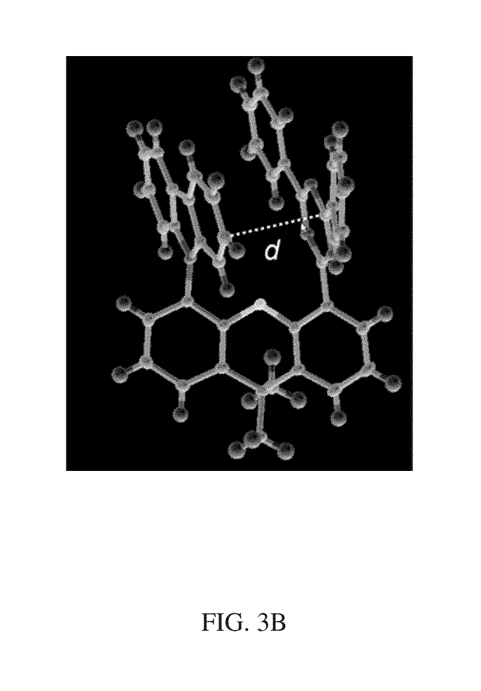

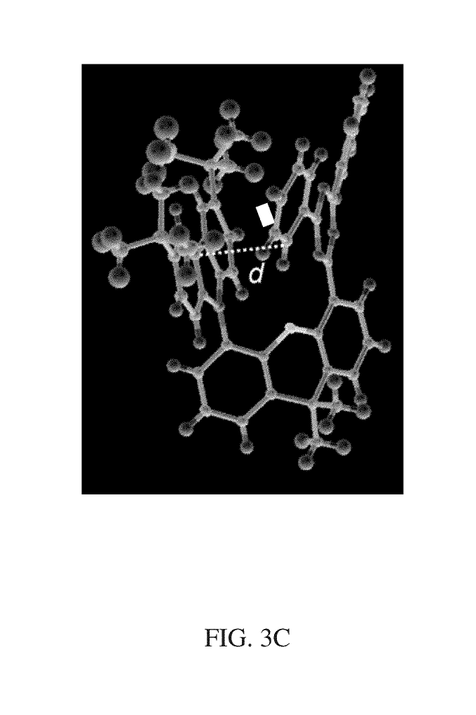

The final products were purified by column chromatography and characterized ¹H-NMR, ¹³C-NMR, high resolution mass spectroscopy, and single crystal structure analysis. The crystal structures shown in FIG. 3A, FIG. 3B, and FIG. 3C confirmed the co-facial arrangement of the donor and acceptor groups at distances (greater than or equal to about 3.3 Angstroms and less than or equal to about 3.5 Angstroms) that were slightly shorter than the calculated geometries (about 4.7 Angstroms). FIG. 3A, FIG. 3B, and FIG. 3C depict the crystal structures of XPT (d=3.423 Angstroms), XCT (d=3.375 Angstroms) and XtBuCT (d=3.299 Angstroms) respectively. The “d” below each structure represents the distances shown by the dashed lines.

### Physical Properties

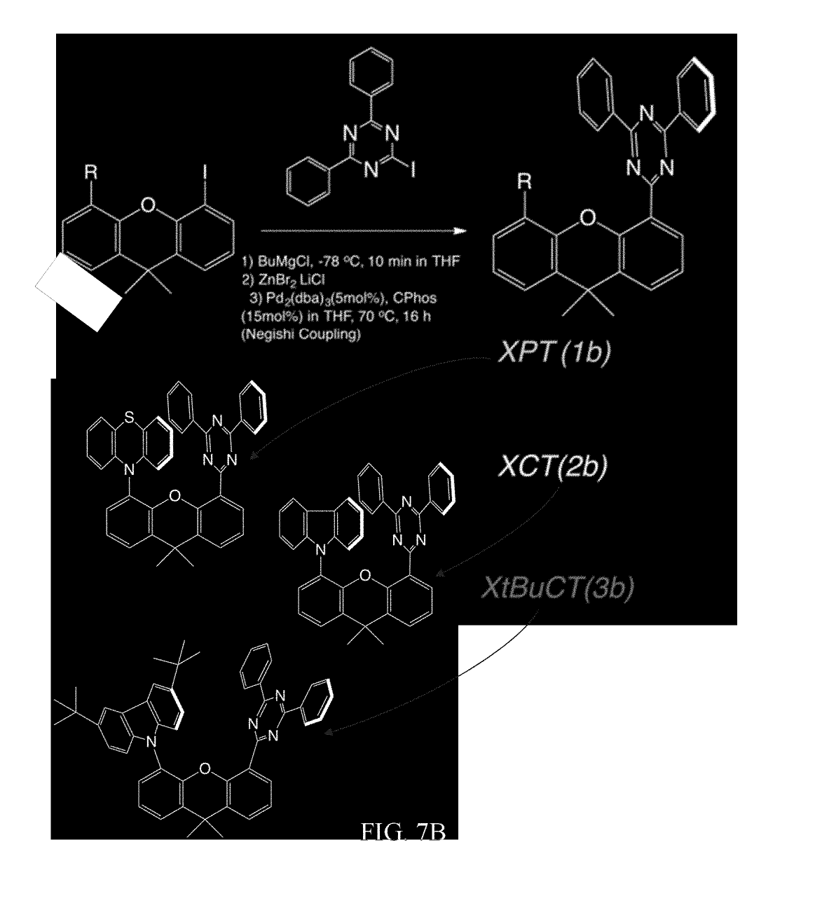

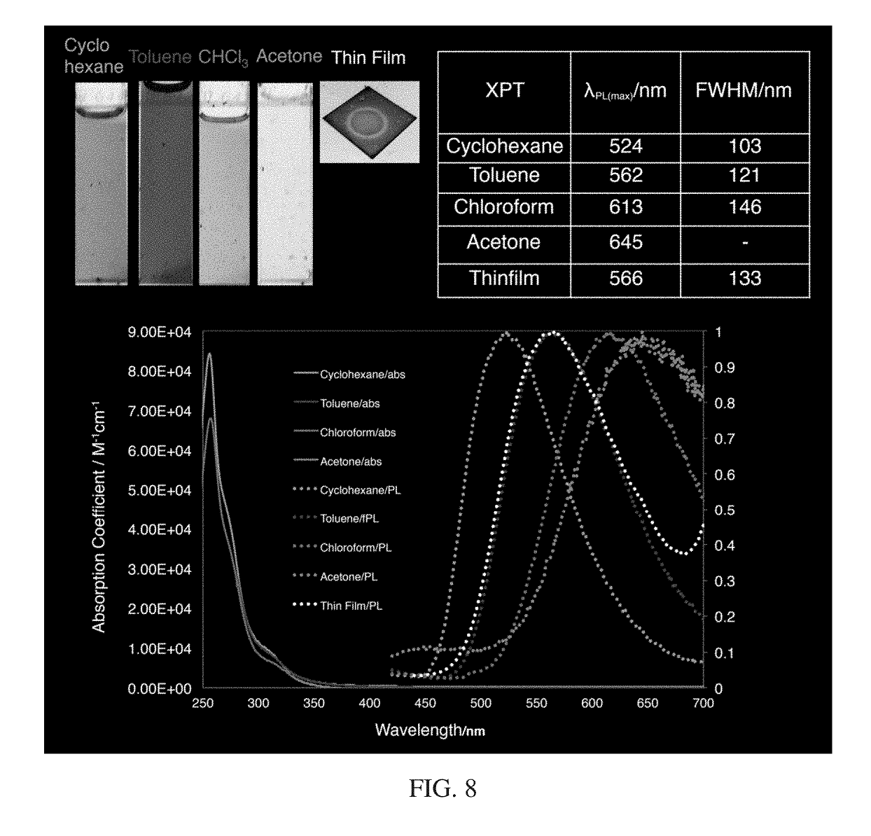

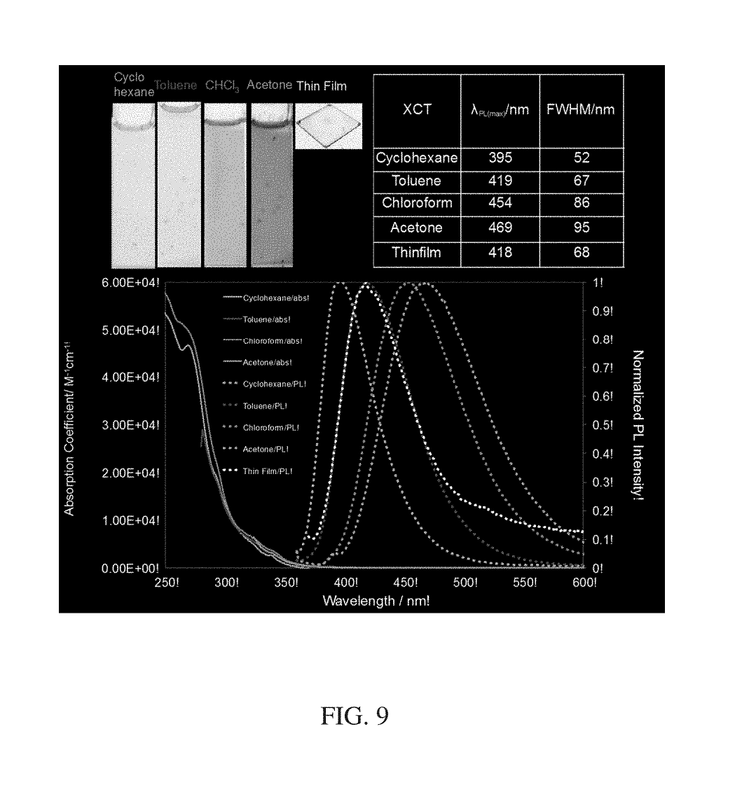

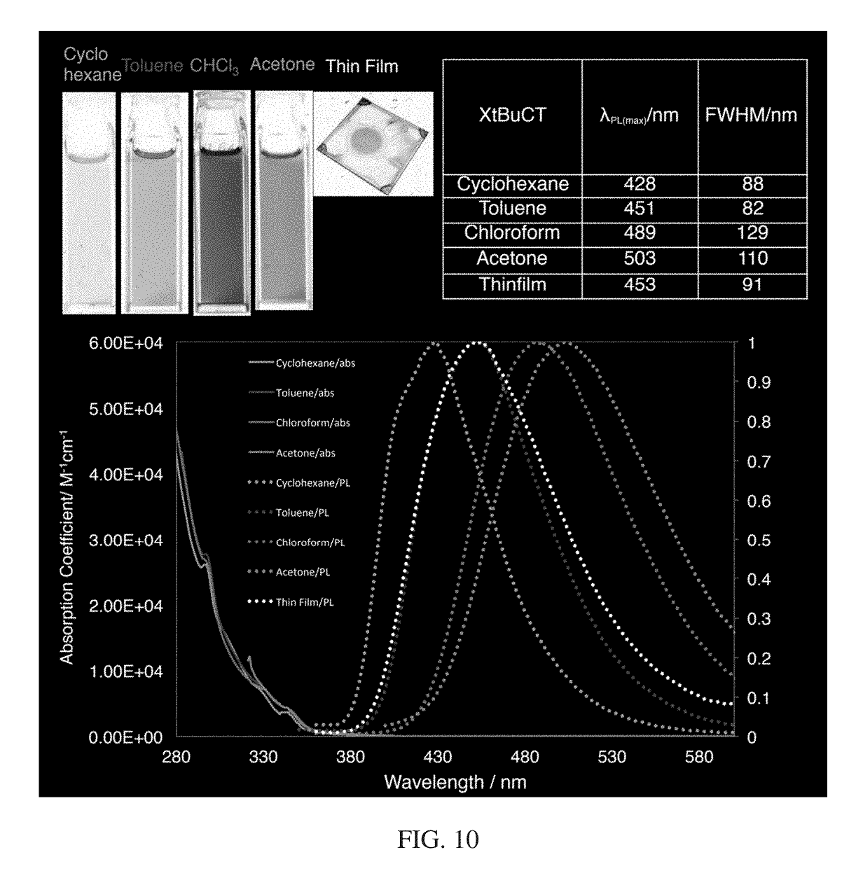

The absorption and photoluminescence spectra of XPT, XCT and XtBuCT are shown in FIG. 8, FIG. 9, and FIG. 10 and summarized in Table 2.

TABLE 2
Physical properties of XPT, XCT and XtBuCT.

| | λ_{abs} | λₑₘ | QY_{sat. O2} | QY_{sat. N2} | τₚ | τ_{d} | λₑₘ |
|---|---|---|---|---|---|---|---|
| dopant | (nm)^{a} | (nm)^{a} | (%)^{b} | (%)^{b} | (ns)^{c} | (μs)^{d} | (nm)^{e} |
| XPT | 272, 310 | 562 | 1.0 | 7.7 | 2.8 | 2.3 | 566 |
| XCT | 293, 341 | 419 | 2.1 | 5.9 | 1.1 | 3.0 | 418 |
| XtBuCT | 298, 346 | 451 | 1.2 | 6.0 | 4.0 | 2.0 | 453 |

| | QY | τ_{d} | T_{g} | T_{g} | HOMO | LUMO | E_{g} |
|---|---|---|---|---|---|---|---|
| dopant | (%)^{f} | (μs)^{g} | (° C.)^{h} | (° C.)ⁱ | (eV)^{j} | (eV)^{k} | (eV)^{l} |
| XPT | 66 | 3.3 | 101 | 318 | −4.99 | −2.22 | 2.77 |
| XCT | — | — | 109 | 312 | −5.58 | −2.39 | 3.19 |
| XtBuCT | 35 | 4.1 | 132 | 313 | −5.49 | −2.33 | 3.16 |

^{a}λ_{abs} is the peak absorption wavelength; λₑₘ is the peak emission wavelength; Measured in toluene (1 × 10⁻⁵ M) at room temperature;
^{b}QY_{sat. O2} is the quantum yield under saturated oxygen; QY_{sat. N2} is the quantum yield under saturated nitrogen; Estimated in toluene using POPOP as the standard (Φ = 0.975 excited at 366 nm in cyclohexane) under saturated O₂ or N₂ at room temperature;
^{c}τₚ is the prompt relaxation time; Measured in toluene using POPOP as the standard (τ = 1.35 ns in ethanol) under saturated O₂ at room temperature;
^{d}τ_{d} is the delayed relaxation time; Measured in toluene (1 × 10⁻⁵ M) under saturated N₂ at room temperature;
^{e}Measured in thin film at room temperature;
^{f}Absolute total quantum yield evaluated using an integrating sphere: 10 wt % dopant doped in DPEPO under N₂ at room temperature;
^{g}Measured in thin film under N₂ at room temperature;
^{h}T_{g} is the glass transition temperature obtained from DSC measurement;
^{I}Obtained from TGA measurement under N₂;
^{j}Estimated from the oxidation potential in CH₂Cl₂ solution by cyclic voltammetry;
^{k}Estimated from HOMO + E_{g}.
Estimated from the onset of absorption spectra in CH₂Cl₂.

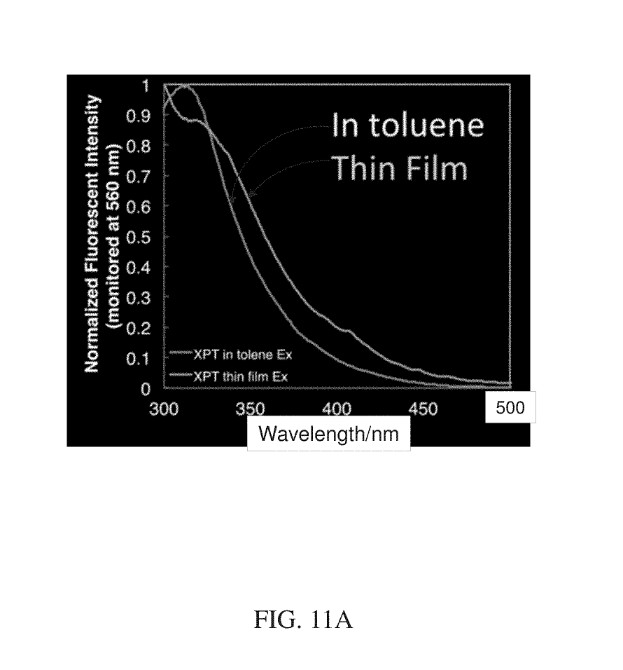

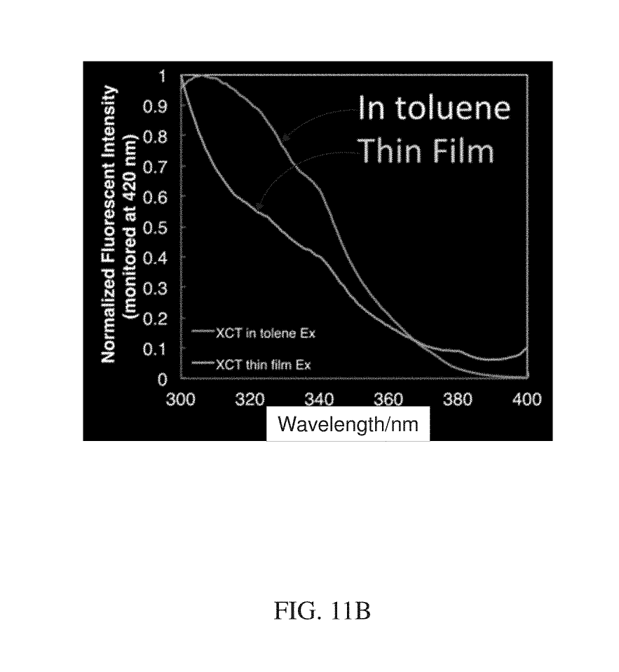

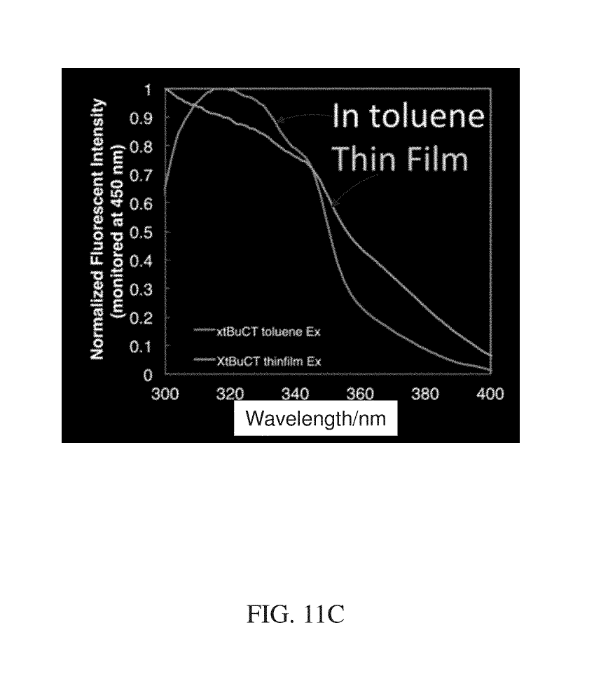

XPT (see e.g., FIG. 8) had a broad absorption band ranging from greater than or equal to about 300 nm to less than or equal to about 360 nm, which was attributed to pi-pi* transitions. Similar broader bands were observed around the same region for XCT (see e.g., FIG. 9) and XtBuCT (see e.g., FIG. 10). The absorption spectra of XPT, XCT and XtBuCT were largely insensitive to the solvent polarity. In contract, photoluminescence (PL) spectra showed strong dependence on solvent polarity. For example, the shift of the XPT emission maximum from 524 nm in cyclohexane to 645 nm in acetone was indicative of a large increase in polarity in the excited state. Consistent with this deduction, the greater the strength of the donor (XPT>XtBuCT>XCT), the greater the red shift from non-polar to polar solvent. XPT exhibited a remarkably large Stokes shift from 214 nm in cyclohexane to 335 nm in acetone (524 nm and 645 nm emission maxima respectively) as a result of its strong intramolecular charge transfer (ICT) character. The excitation spectra of the molecules showed maxima at 313 nm for XPT, 306 nm for XCT and 317 nm for XtBuCT in toluene. Conversely, the thin film forms gave less defined excitation spectra with larger intensity at lower wavelengths, which represented a composite of direct excitation and energy transfer at lower wavelengths (see e.g., FIG. 11A, FIG. 11B, and FIG. 11C).

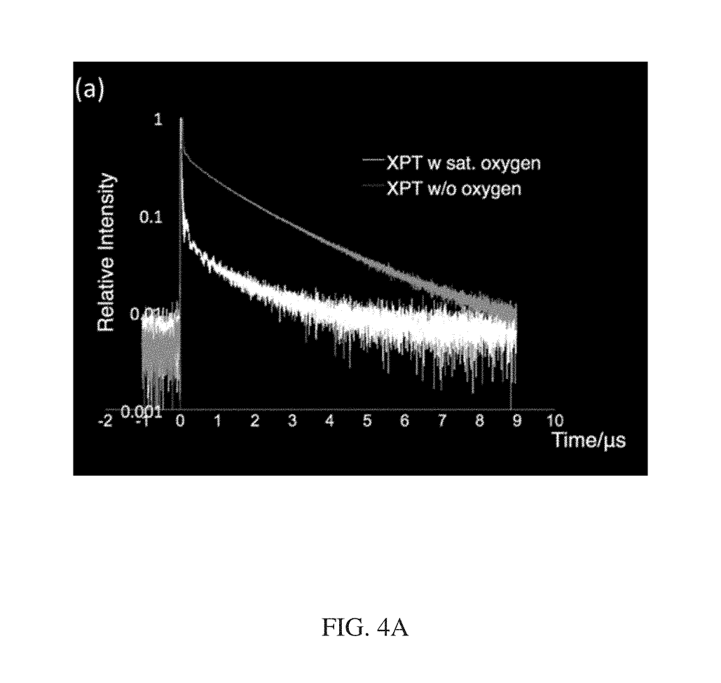

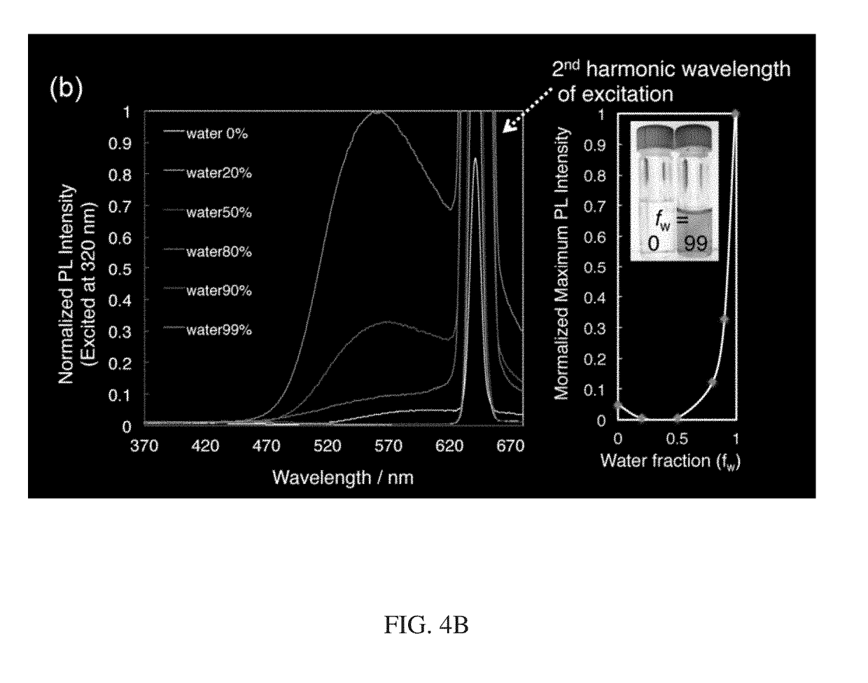

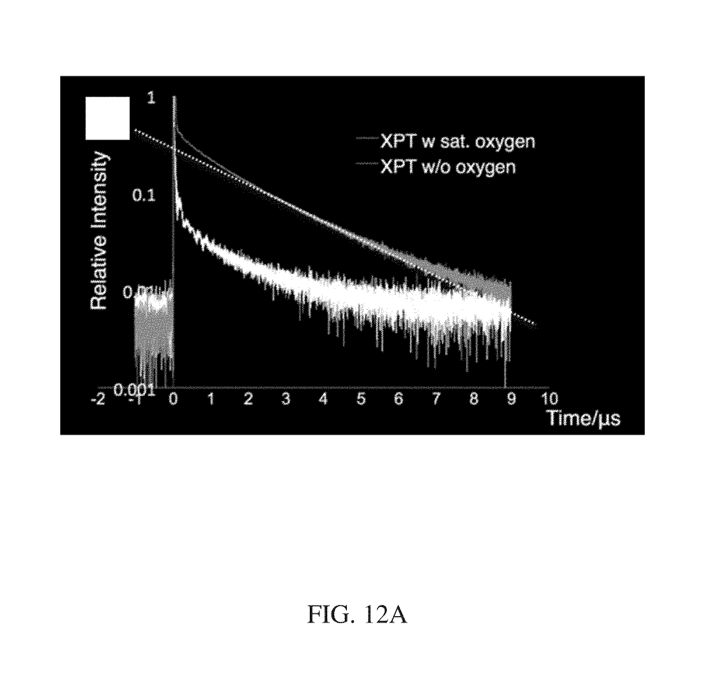

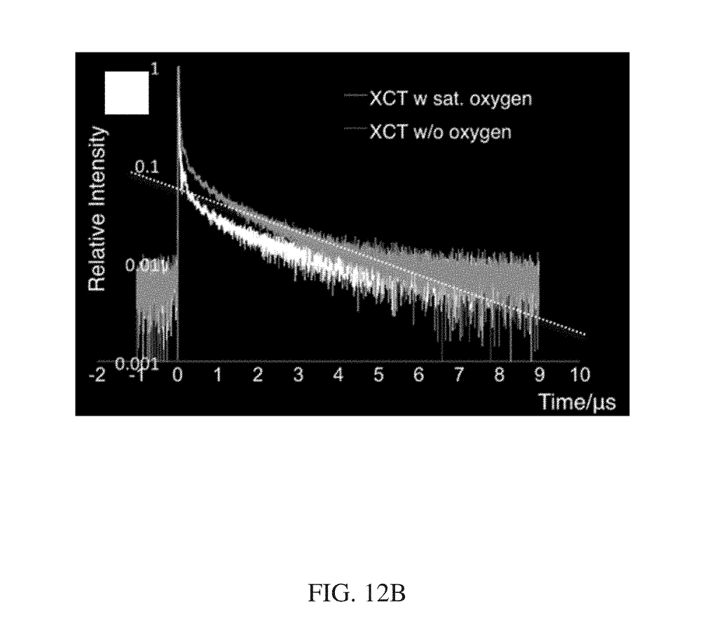

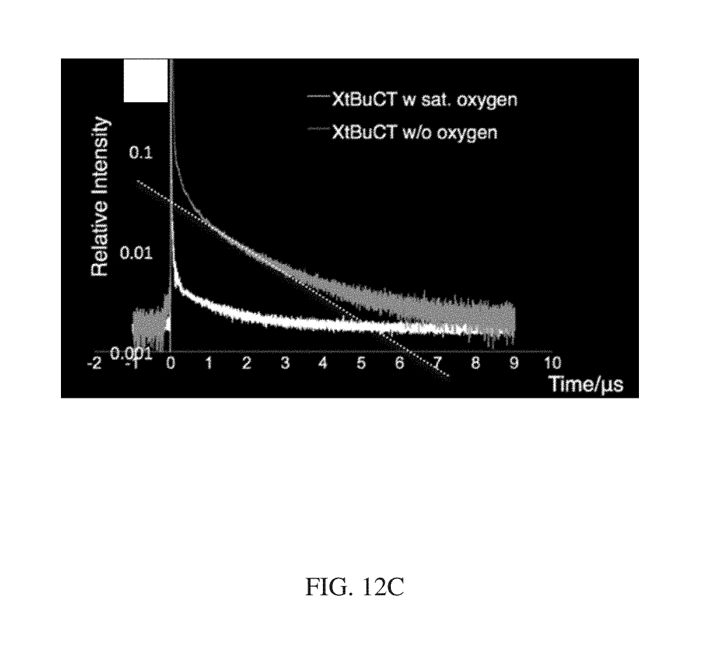

To probe the anticipated small energy gap of the T₁ and S₁ levels, photoluminescence quantum yields (PLQYs) and excited state lifetimes (τ) were measured in the presence and absence of triplet-quenching oxygen in toluene (see e.g., Table 1, FIG. 4A, FIG. 12A, FIG. 12B, and FIG. 12C).

FIG. 4A shows a PL transient spectrum of XPT in toluene under saturated oxygen and saturated nitrogen at room temperature; the concentration of XPT was 1×10⁴, and λₑₓ=336 nm. FIG. 4B shows PL spectra of XPT in a THF/water mixture and the change of normalized PL peak intensity with different water fractions; the concentration of XPT was 1×10⁻⁴ M, λₑₓ=320 nm; inset: PL images of XPT with different water fractions under 365 nm UV light.

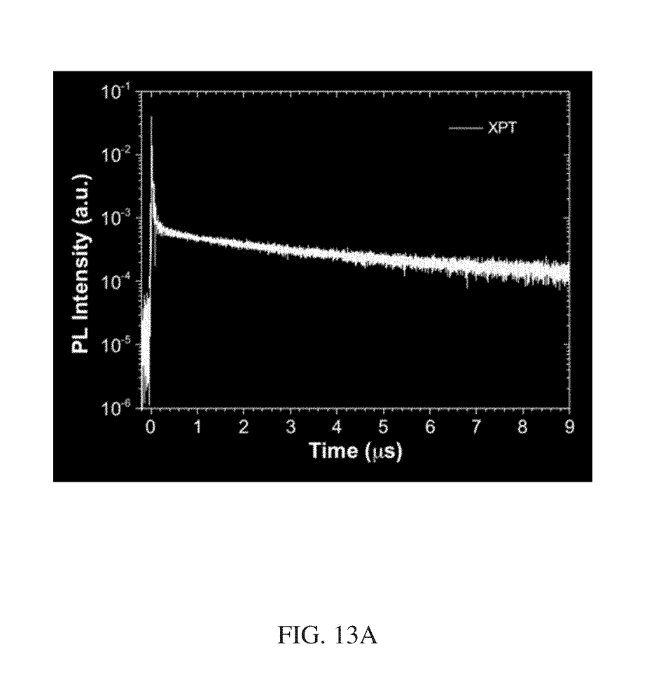

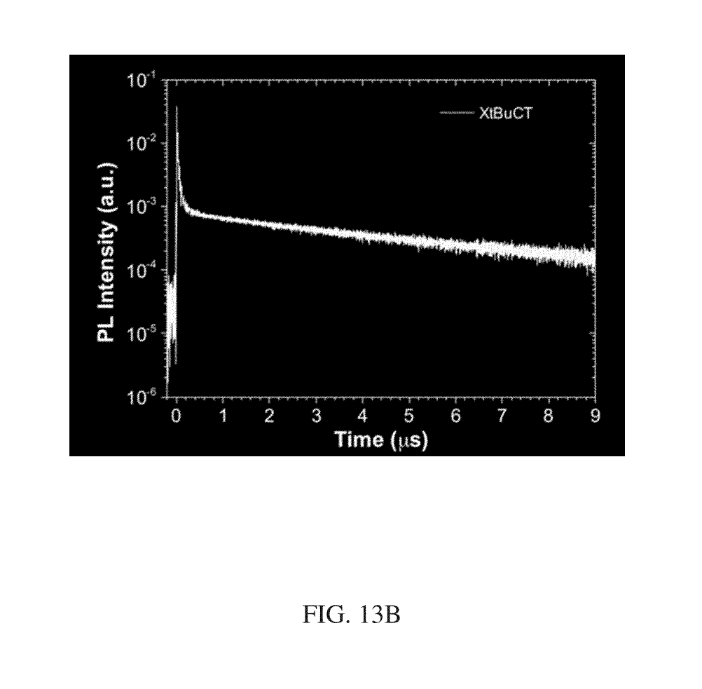

When oxygen was excluded, the PLQYs increased, with XPT having increased from 1.0% to 7.7%, XCT having increased from 2.1% to 5.9%, and XtBuCT having increased from 1.2% to 6.0%. Additionally, higher quantum yields were observed with XPT and XtBuCT from 7.7% to 65% and 6.0% to 35% in nitrogen-bubbled toluene and in thin film under nitrogen, respectively. Under nitrogen, XPT displayed distinctive delayed (τ_{d}) 2.3 microseconds relaxation in addition to a prompt (τₚ) 2.8 nanosecond relaxations. Similar relaxations were observed for XCT (τₚ=1.1 nanoseconds, τ_{d}=3.0 microseconds) and XtBuCT (τₚ=4.0 nanoseconds, τ_{d}=2.0 microseconds). After bubbling with oxygen, the delayed components were substantially decreased or not detectable. The delayed fluorescence was also observed in the solid state (τ_{d}=3.3 microseconds for XPT, τ_{d}=4.1 microseconds for XtBuCT) (see e.g., FIG. 13A, FIG. 13B). These results with prompt and delayed emissive components indicated TADF behavior.

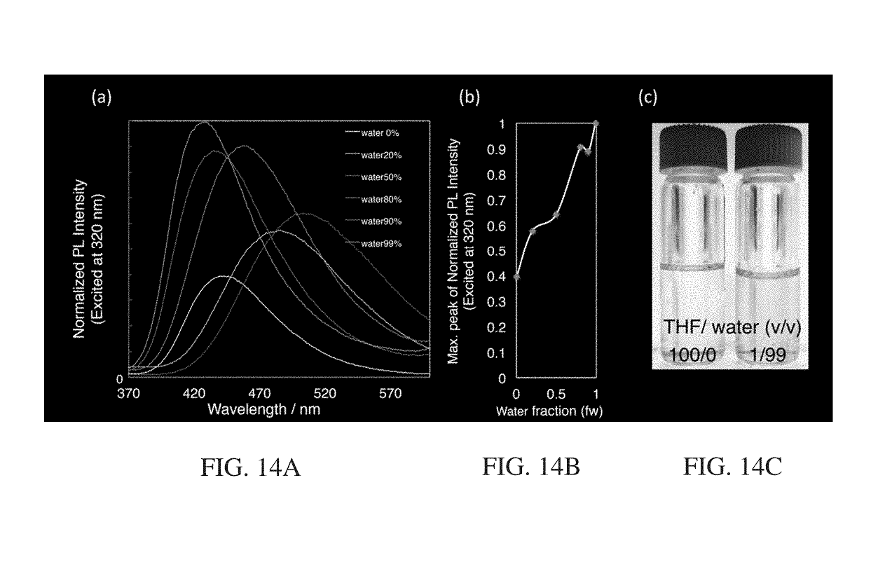

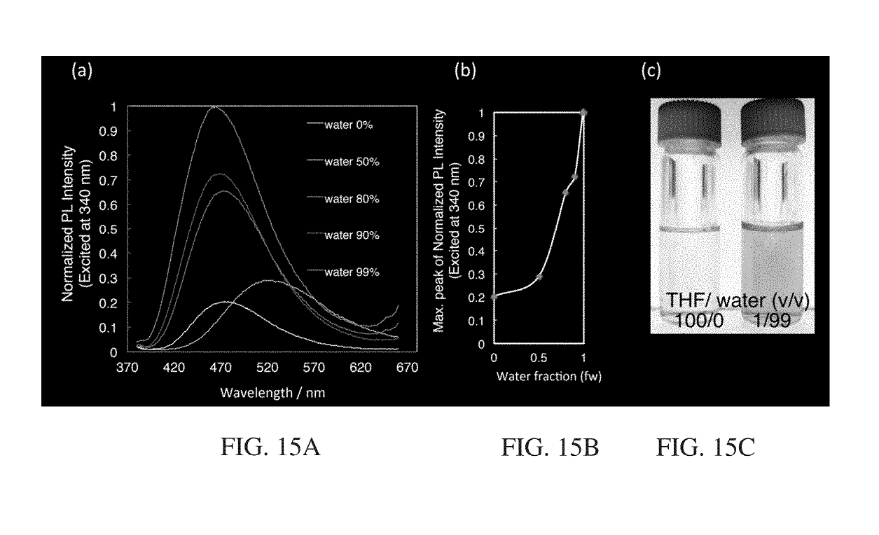

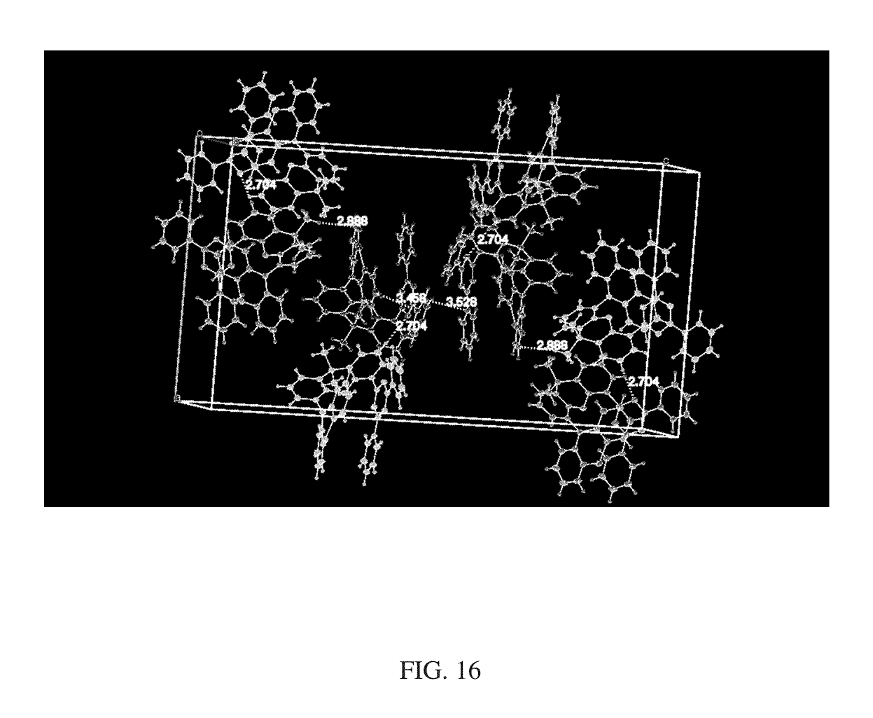

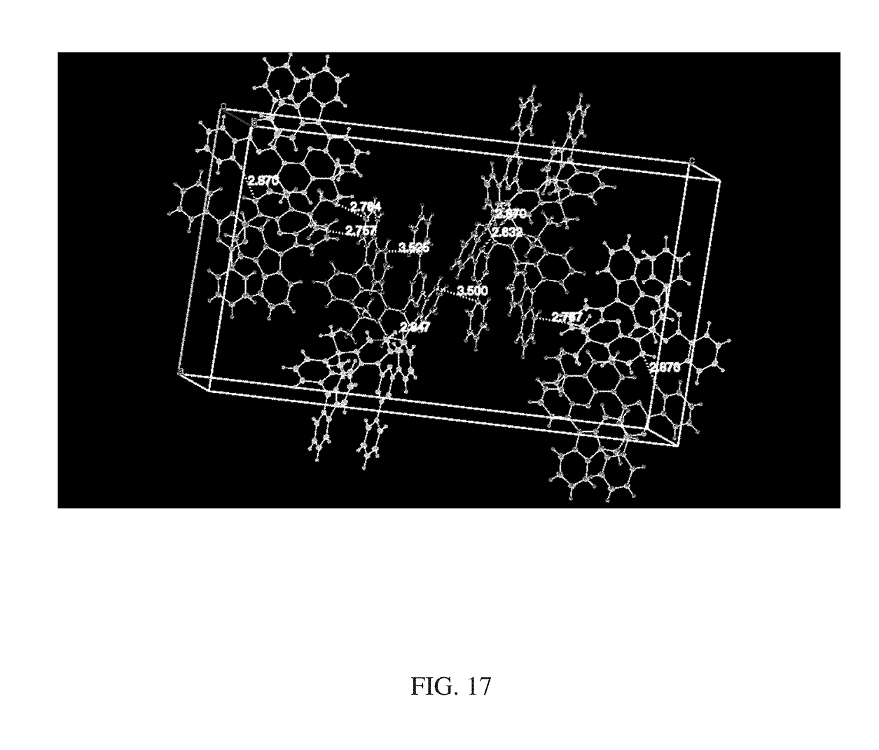

The xanthene molecules incorporated with donor-acceptor exhibited lower PLQY in solution and showed an increase emission efficiency in solid state, a process that is known as aggregation induced emission (AIE). The AIE phenomenon can result in highly emissive materials. Additionally, AIE can be effective at minimizing singlet-triplet and triplet-triplet annihilation in OLED systems and therefore, these AIE-active TADF (AIDF) materials were an attractive strategy to achieve high IQE OLED devices. To investigate AIE in these materials, water was added to tetrahydrofuran (THF) solutions as indicated in FIG. 4B. For XPT, a relative PL intensity was 0.05 in THF, then the intensity of the PL was reduced and red shifted at about 50% by weight water (f_{w}). At this stage, XPT was still dissolved and an increase in the charge transfer character lowered the quantum yield. Increasing f_{w} to 80% produced a dramatic increase in a blue-shifted PL, and for higher f_{w}'s the PL continued to increase, with it being 20 times higher than the initial THF solution (f_{w}=99). At such large water contents, nano-clusters with less polar environments formed and the emission blue-shifted and the restricted motion lowered the dynamics of the system and attenuated competing non-radiative transitions. Similar, but less dramatic, AIE behaviors (see e.g., FIG. 14A, FIG. 14B, FIG. 14C, FIG. 15A, FIG. 15B, FIG. 15C) were observed for XCT and XtBuCT that contained higher rigidity carbazole rings. The crystal packing of XPT suggested two possible modes of intermolecular interactions and/or intramolecular interactions including intramolecular pi-pi and intermolecular C-H . . . pi interactions (see e.g., FIG. 16). The XPT had an intramolecular pi-pi interaction between the donor and acceptor at greater than or equal to about 3.3 Angstroms and less than or equal to about 3.5 Angstroms, which allowed for efficient charge transfer (CT) without producing a strong ground state pi interaction. Additionally, intermolecular C-H . . . pi interactions were observed in a unit cell at the distance of greater than or equal to about 2.7 Angstroms and less than or equal to about 2.9 Angstroms that stabilized the crystal packing and restricted the intramolecular motion, giving rise to AIE. The crystal packing structures of XCT and XtBuCT also showed intermolecular C-H . . . pi interactions that could assist in creating AIE behavior (see e.g., FIG. 17, FIG. 18).

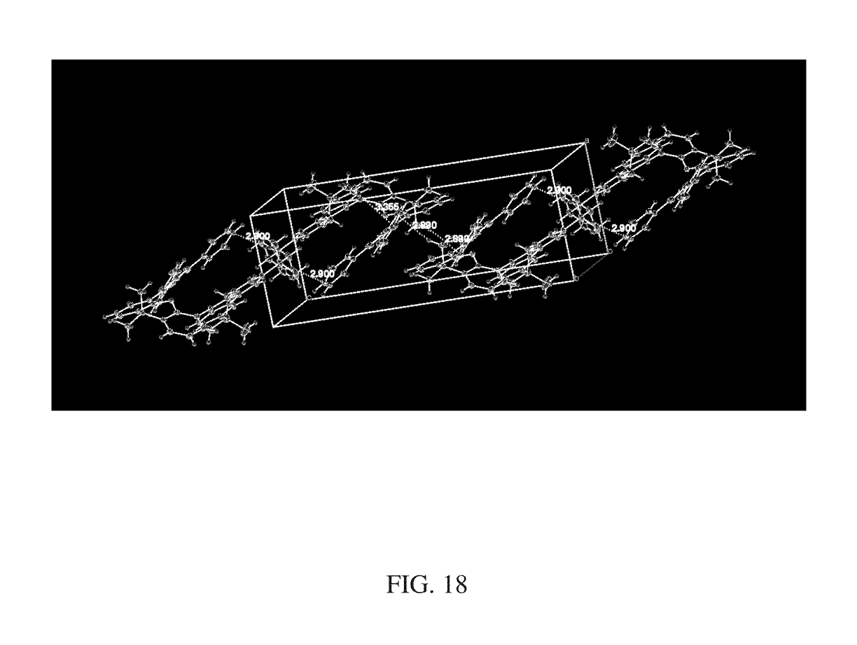

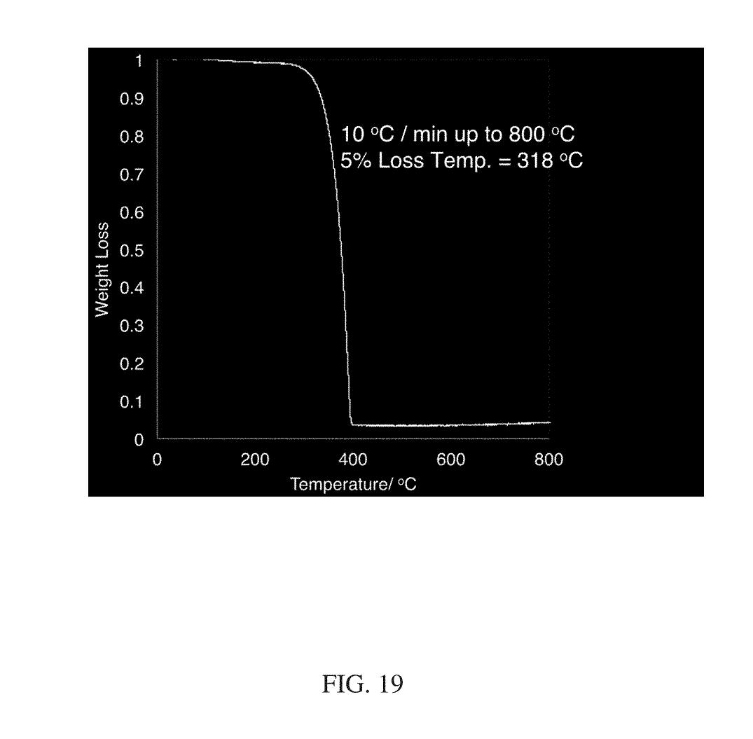

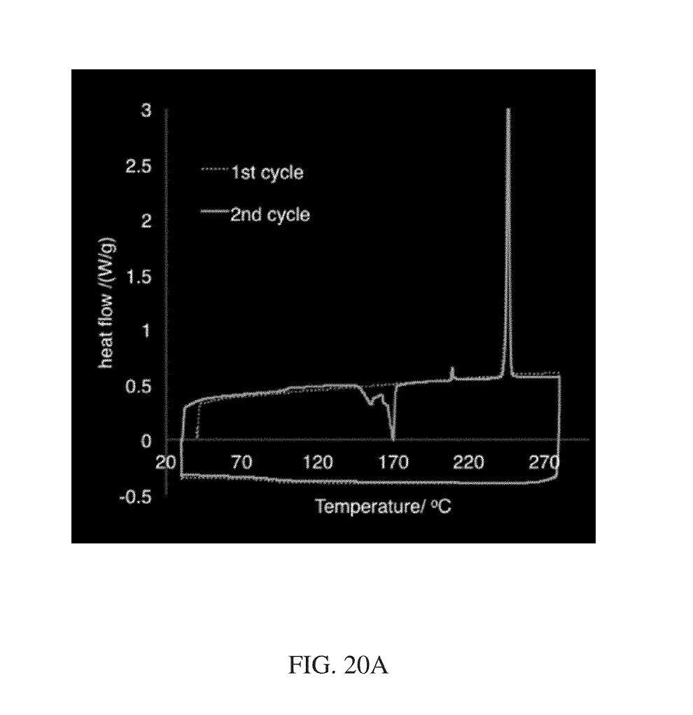

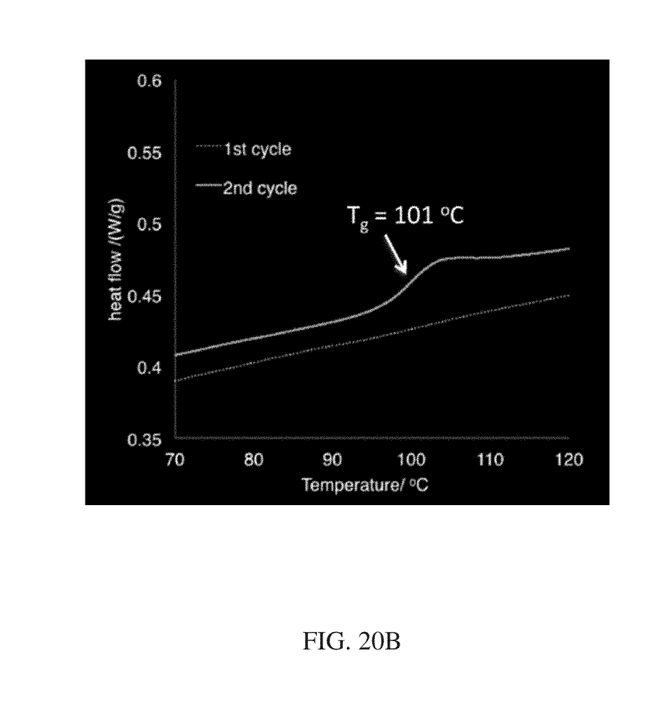

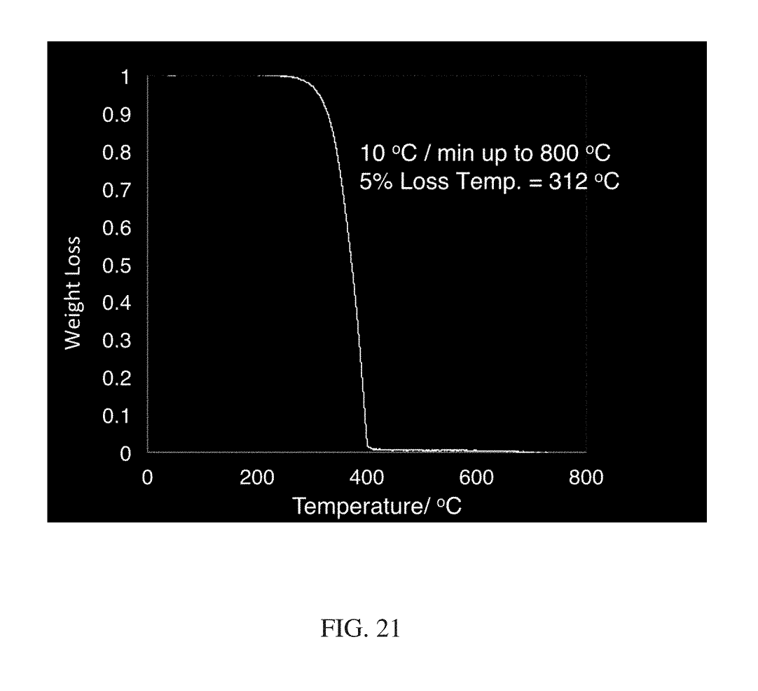

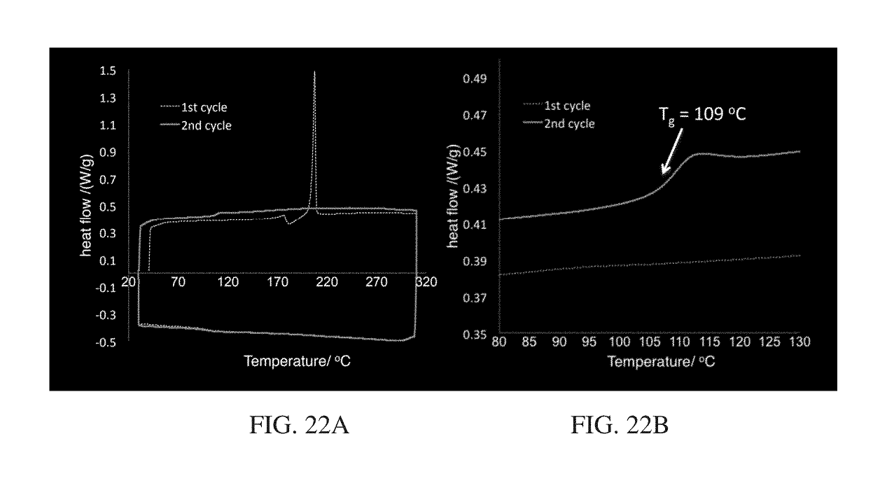

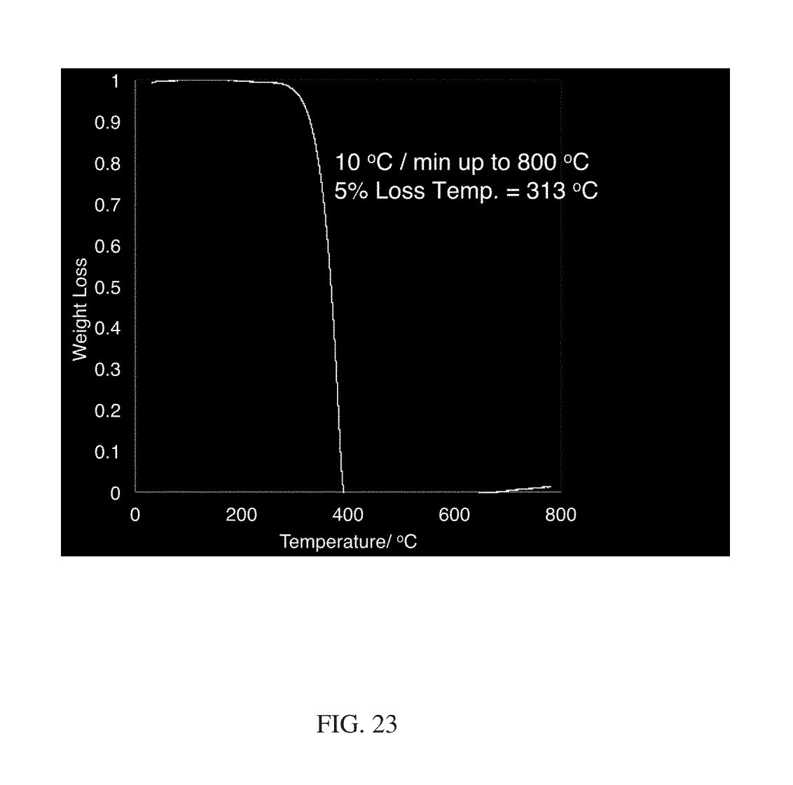

The thermal properties of XPT, XCT and XtBuCT were investigated by thermogravimetric analysis (TGA) and differential scanning calorimetry (DSC) (see e.g., FIG. 19, FIG. 20A, FIG. 20B, FIG. 21, FIG. 22A, FIG. 22B, FIG. 23, FIG. 24A, FIG. 24B). Thermal stability helps to create stable films by chemical vapor deposition, and 5% weight loss was measured at temperatures greater than or equal to about 310 degrees Celsius and less than or equal to about 320 degrees Celsius and the materials displayed glass transition temperatures (T_{g}) greater than or equal to about 101 degrees Celsius and less than or equal to about 132 degrees Celsius. XtBuCT showed the highest T_{g}, which was attributed to the di-tert-butyl group.

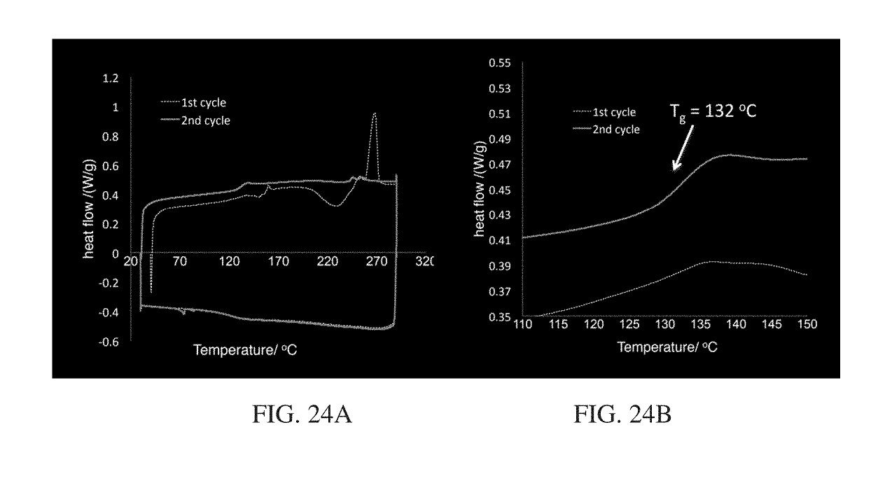

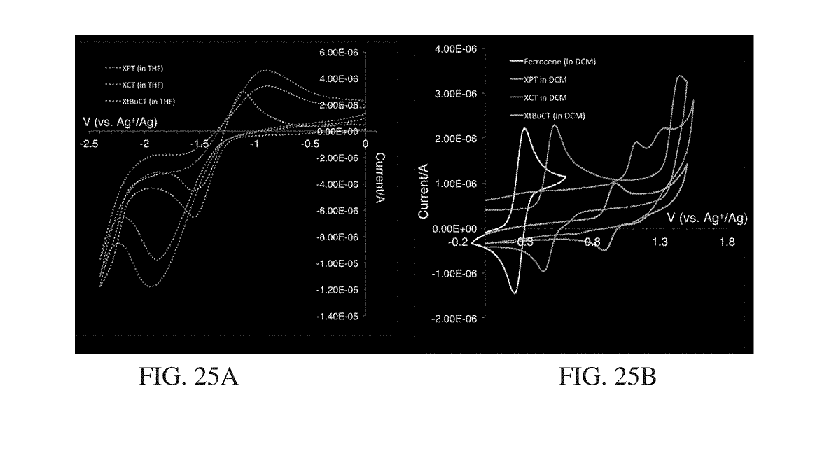

Cyclic voltammetry was performed to determine the relative oxidative energies of our xanthene TDAF materials (see e.g., FIG. 25A, FIG. 25B) and the results are summarized in Table 1 and Table 3.

TABLE 3

| | E^{1/0}_{1/2}(E^{onset})/eV | | E^{0/-1}_{1/2}(E^{onset})/eV | E_{HOMO}/eV | E_{LUMO}/eV | E_{g}^{elec}/eV | E_{g}^{opt}/eV | E_{g}^{calc}/eV |
|---|---|---|---|---|---|---|---|---|
| XPT | 0.47 | (0.49) | −1.40 (−1.35) | −4.99 | −3.15 | 1.84 | 2.77 | 3.108 |
| XCT | irev | (1.03) | −1.43 (−1.31) | −5.58 | −3.12 | 2.46 | 3.19 | 3.614 |
| XtBuCt | 0.94 | (0.89) | −1.33 (−1.30) | −5.49 | −3.22 | 2 27 | 3.16 | 3.438 |

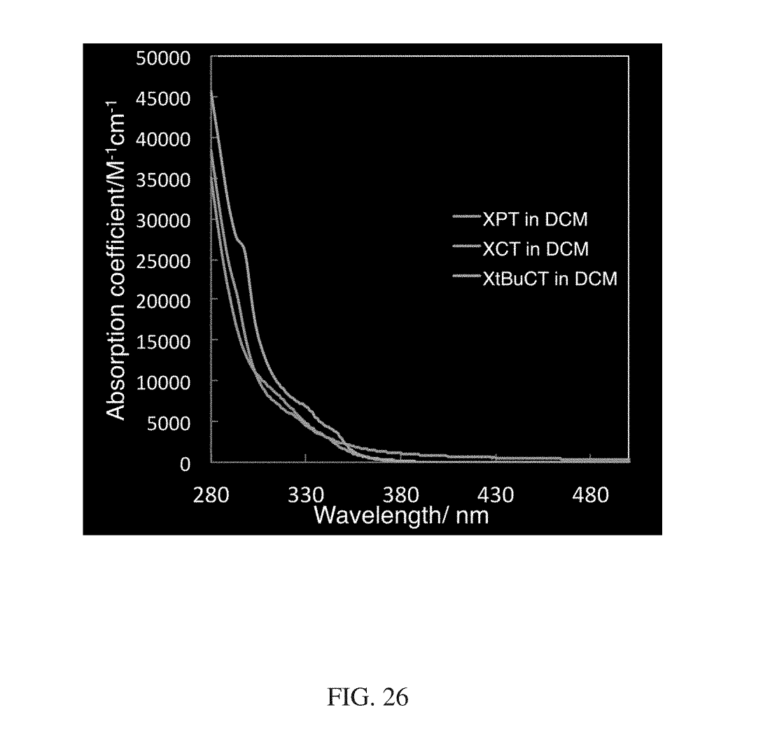

Based upon the oxidative waves in anodic sweep, the electrochemical HOMO levels were estimated to be −4.99 eV, −5.58 eV and −5.49 eV for XPT, XCT and XtBuCT respectively. HOMO levels were decreased (XCT>XtBuCT>XPT) which was consistent with the electron-donating characteristics (phenothiazine>di-tert-butylcarbazole>carbazole). XCT showed irreversible oxidative waves and was less stable than XtBuCT and XPT, as a result of oxidative coupling at the 3- and 6-positions that were para to the nitrogen atom of the carbazole. The optical bandgaps (E_{g}) were determined from the onset of the absorption band (see e.g., FIG. 26) in CH₂Cl₂ to be 2.77 eV, 3.19 eV, and 3.16 eV, and LUMO energy levels were then estimated from HOMO minus E_{g} to be 2.22 eV, 2.39 eV, and 2.33 eV for XPT, XCT and XtBuCT respectively.

### Through-Space Charge-Transfer

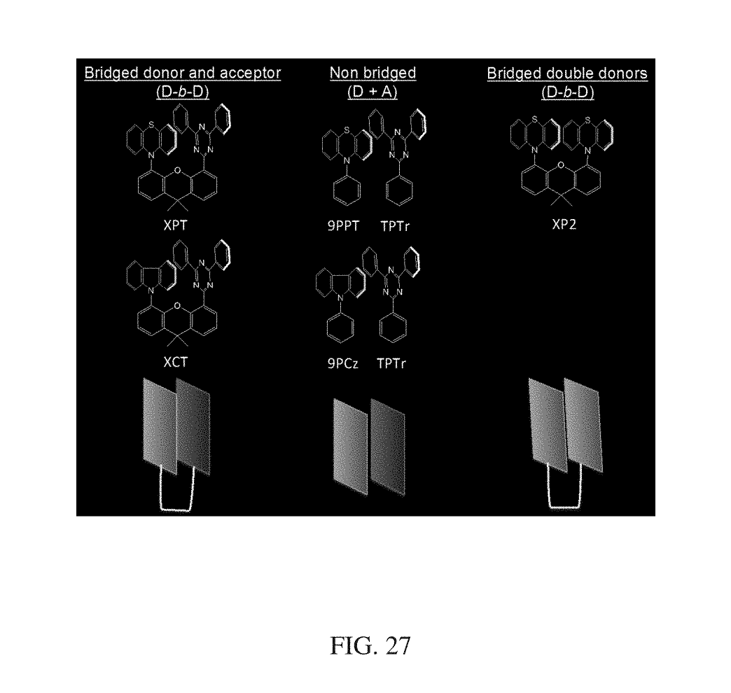

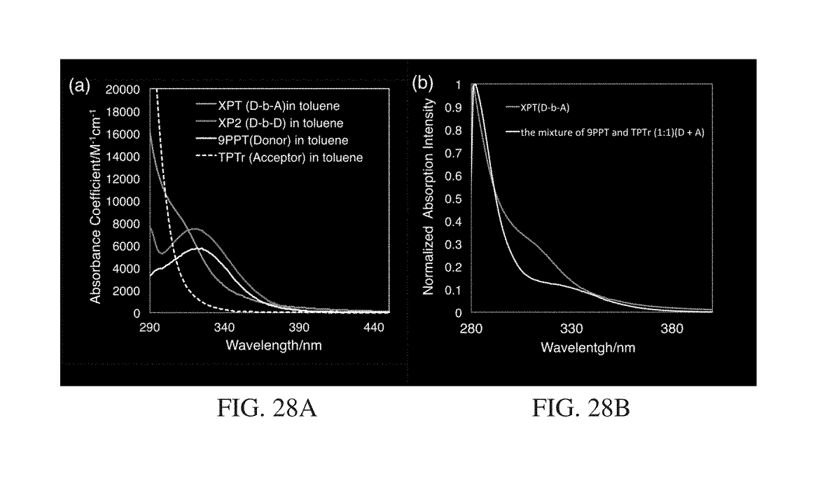

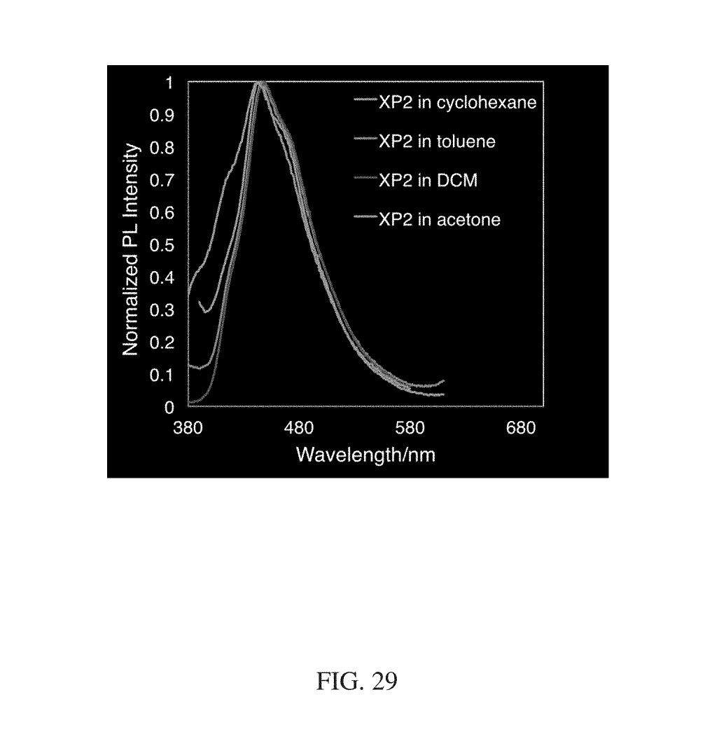

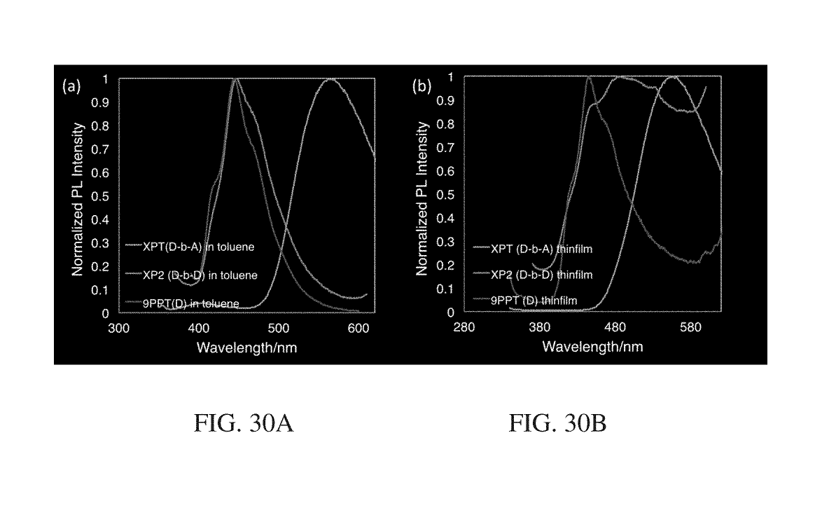

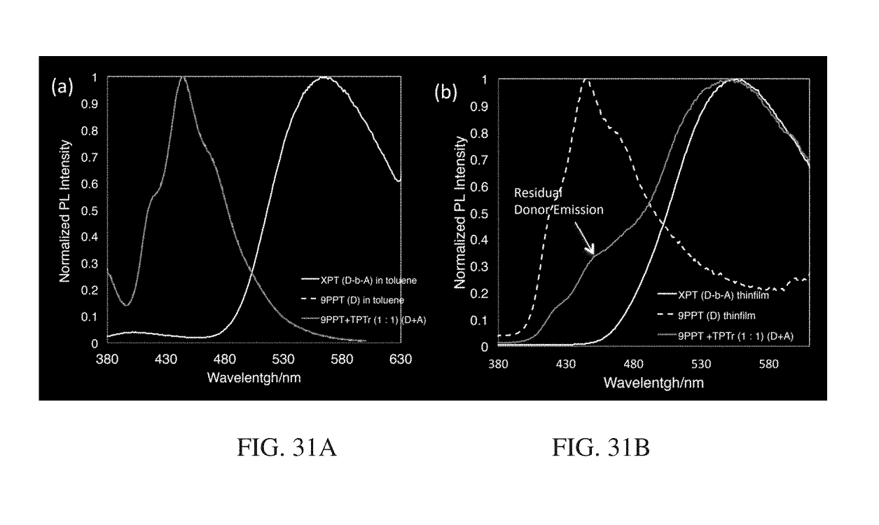

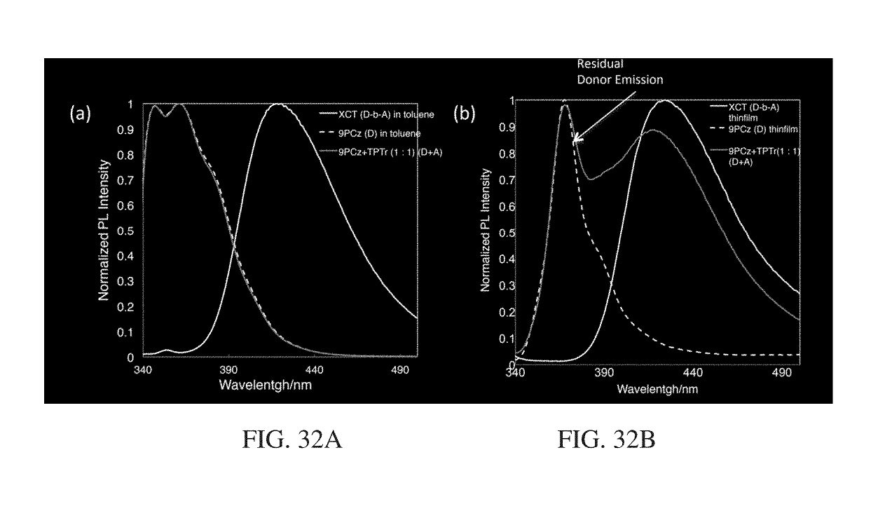

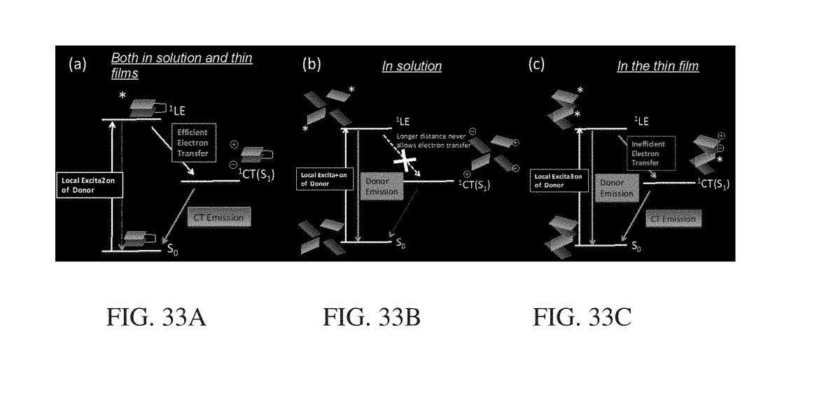

Previous efforts to create D-A exciplex TADF emitters (see e.g., FIG. 1A, 120) generally resulted in D-A exciplex structures typically displaying lower EQEs than the twisted D-A single TADF molecules. These D-A exciplex systems had very small DELTA_E_{ST} (usually less than 50 meV), but co-evaporation of donor and acceptor molecules led to a distribution of structures with uncontrolled intermolecular interactions and low EQEs. By contrast, in the current example, excited state through-space charge-transfer (CT) was produced in a single molecule. To probe the importance of the intermolecular character, control experiments were conducted using different architectures. Specifically, bridged donor acceptor molecules (D-b-A) XPT and XCT were compared with mixtures of model donors (D) 9-phenylphenothiazine (9PPT) or 9-phenylcarbazone (9PCz) and the model acceptor (A) 1,3,5-triphenyl-2,4,6-triazine (TPTr). The bridged donor-donor (D-b-D) molecule XP2 (FIG. 27) was further studied. The absorption spectra of XPT, XP2, 9PPT, and TPTr are shown in FIG. 45A and FIG. 45B. XP2 and 9PPT exhibited similar absorption spectra and had absorption maxima at around 320 nm, which were attributable to pi-pi* transition of the phenothiazine group. TPTr didn't have a specific absorption over this region. On the other hand, XPT showed a distinctive broad absorption band greater than or equal to about 300 nm and less than or equal to about 330 nm, which was attributable to a D-A interaction. XP2 exhibited PL around 445 nm in various polar environments, and as expected for the D-b-D structure, it did not show solvatochromism (FIG. 29). PLs of XPT (D-b-A) and XCT (D-b-A), XP2 (D-b-D), 9PPT (D), and 9PCz (D) in toluene and thin film are shown in FIG. 30A and FIG. 30B. In toluene, XPT (D-b-A) had PL with a maximum at 562 nm, which was attributable to CT emission and more red-shifting than PL of XP2 (D-b-D) or 9PPT (D) with a maximum at 445 nm. TPTr (A) was not emissive, and was therefore absent in these spectra. In thin films, similar behavior was observed; however, XP2 (D-b-D) was less emissive and showed a slightly more red-shifted PL at 487 nm than that of 9PPT (D), suggesting pi-pi aggregation. A 1:1 mixture of 9PPT (D) and TPTr (A) in toluene displayed an emission maximum at 445 nm which was assignable to a 9PPT (D) emission. Conversely, spin-coated thin films of 1:1 9PPT (D) and TPTr (A) had a PL maximum at 552 nm with a shoulder at 445 nm, which were attributable to CT excimer emission (¹CT) and localized donor emission (¹LE) respectively (see e.g., FIG. 31A, FIG. 31B). The same behavior was observed for XCT (D-b-A) in comparison to solutions and thin films containing mixtures of 9PCz (D) and TPTr (A) (see e.g., FIG. 32A, FIG. 32B). This behavior revealed (FIG. 33A, FIG. 33B, FIG. 33C) that the 9,9-dimethylxanthene bridged chromophores only displayed the CT excimeric emission independent of the concentrations and solvent; however, the excimers between the model D and A units were not formed in solution. In thin films, the donor and acceptor constituents formed excimers but the organization was not sufficient to prevent some residual emission from the D constituents.

### Mechanochromism

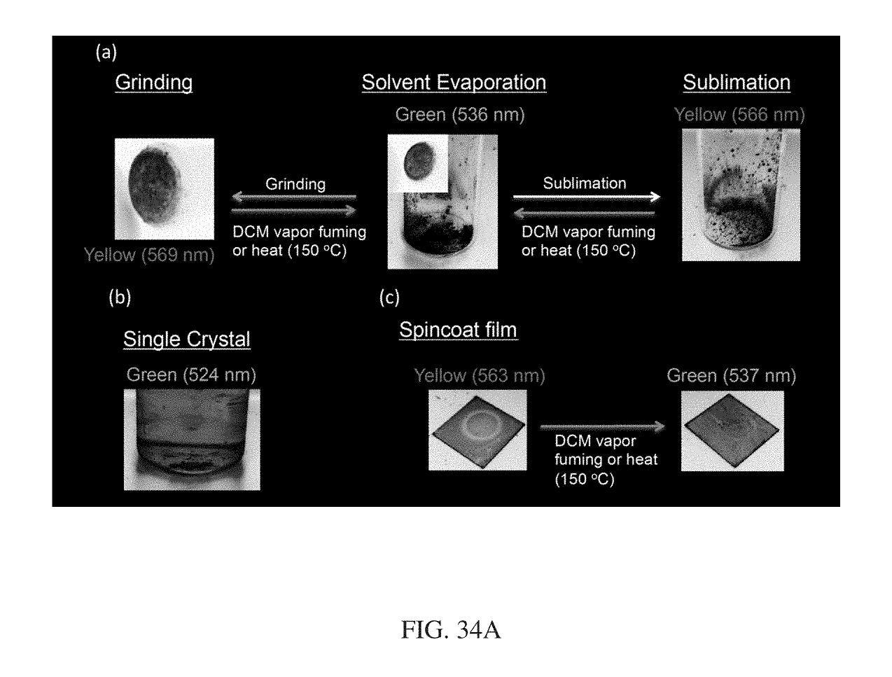

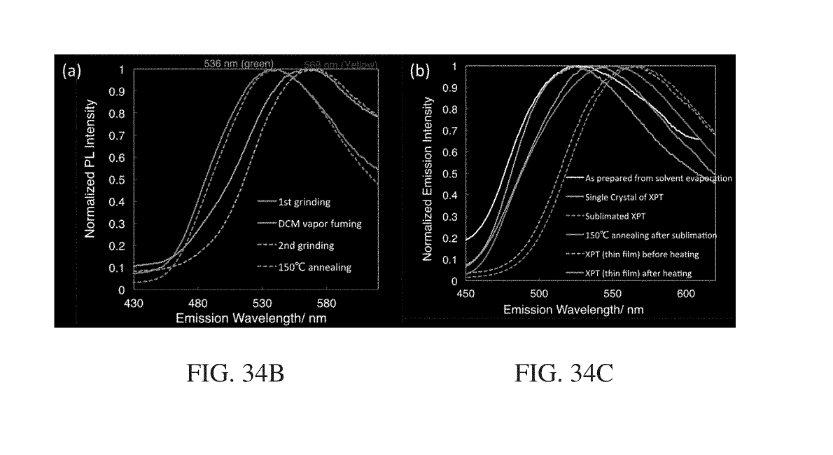

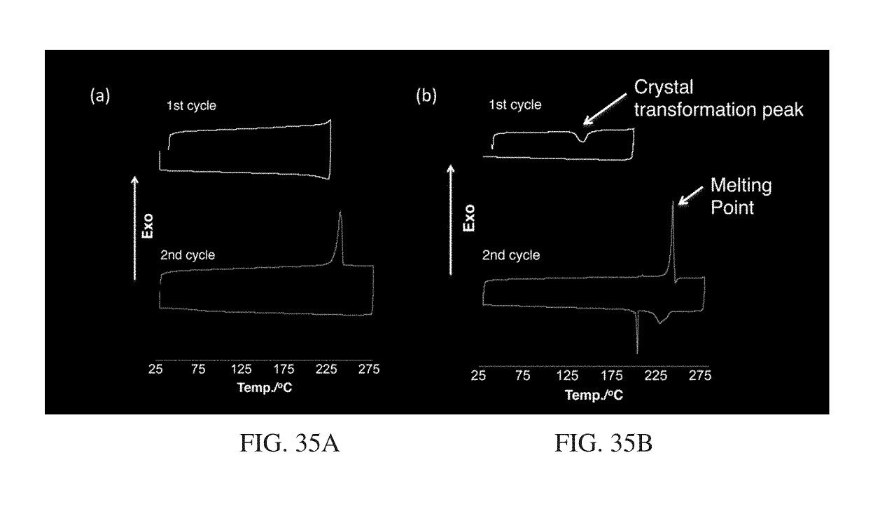

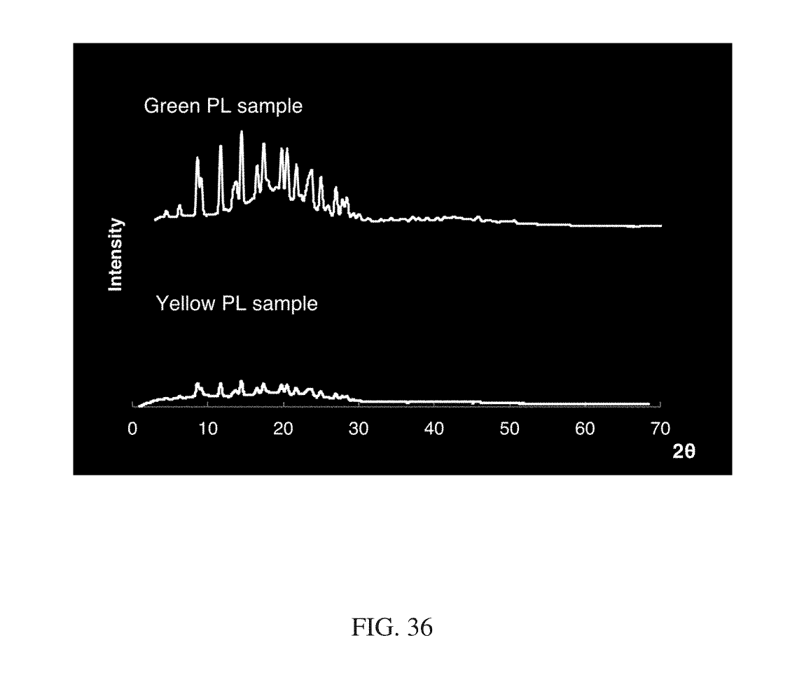

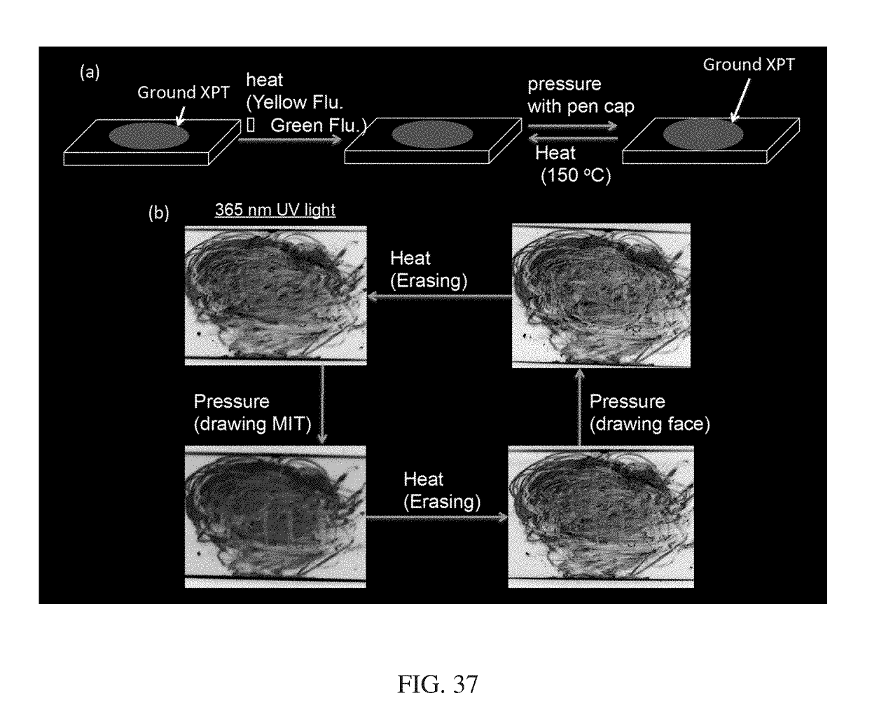

XPT displayed notable mechanochromic properties. A single crystal of XPT obtained from vapor diffusion (pentane, in the presence of 1,2-dichlorobenzene vapor) technique exhibited green PL at 524 nm. However, crystals produced from solvent evaporation gave a slightly red-shifted emission maximum at 536 nm. Upon grinding with a pestle and mortar, the emission and excitation spectra changed and the emission became yellow with a maximum PL at 569 nm (FIG. 34A). The 536 nm green emission was restored by directing CH₂Cl₂ vapor at the sample or heating over the T_{g} temperatures previously determined. These processes were all reversible and the samples was able to be subjected to multiple cycles. Sublimated or spin-coated films of XPT displayed emission spectra similar to that of the ground solid (λₑₙ, =563-569 nm) (FIG. 34B) and after exposure to CH₂Cl₂ vapor or heating over T_{g}, the green emission at 536 nm was generated. Based upon this behavior the green emission was concluded to be more stable, and DSC profiles of two different states are shown in FIG. 35A and FIG. 35B. Clearly, the green material was a single phase and showed only a melting transition. Upon initial heating, the yellow ground phases had a metastable state that underwent an exothermic crystallization transition at about 130 degrees Celsius in the first heating, which was associated with the transition to the green emitting phase. Once thermally equilibrated, both materials had the same melting points. Powder XRD did not reveal any new sharp reflexes for the yellow solid that were not present in the green solid (FIG. 36). Hence, the DSC and XRD investigations suggested that the yellow metastable phases were disordered or amorphous. Using ground XPT, reversible drawing and erasing could be readily performed by placing materials on a glass substrate (FIG. 37). The yellow ‘MIT’ or face images were written with pressure. Upon heating, yellow ‘MIT’ characters were completely erased and this reversible process was able to be conducted in succession many times.

### Device Fabrication and Evaluation

Having observed the AIDF properties of the D-A xanthene molecules in this example, OLED devices comprising the D-A xanthene molecules were fabricated and evaluated. XPT and XtBuCT were used as emissive dopants in OLED devices created by thermal evaporation with the following architecture (FIG. 5A): ITO (100 nm)/1,1-bis[4-[N,N′-di(p-tolyl)amino]-phenyl] cyclohexane (TAPC) (70 nm)/oxybis(2,1-phenylene))bis-(diphenylphosphine oxide) (DPEPO): Dopant (10%) (30 nm)/DPEPO (2 nm)/1,3,5-tri(m-pyrid-3-yl-phenyl)benzene (TmPyPb) (45 nm)/LiF (1 nm)/Al (100 nm). FIG. 5A shows OLEDs device structure used in this example, in which the dopant was at least one of XPT, XCT, and XtBuCT. FIG. 5B shows current density-voltage-luminance characteristics of devices fabricated using XPT and XtBuCT. The XPT device showed a lower turn-on voltage than the XtBuCT device as a result of its lower HOMO-LUMO gap. The electroluminescence (EL) spectra (FIG. 5C) displayed peaks at 584 nm for XPT devices and 488 nm for XtBuCT devices that were red-shifted relative to the PL determined in toluene solution. Considering the solvatochromic properties of these molecules, this was likely at least in part caused by the polar nature of DPEPO. FIG. 5C shows EL spectra of devices fabricated using XPT and XtBuCT at 1 mA/cm². FIG. 5D shows the EQEs of OLEDs fabricated using XPT and XtBuCT as a function of current density. As shown in FIG. 5D, the maximum EQE of XPT devices was 10%, which exceeded the theoretical limit of 6% expected for a simple fluorescent OLED. Therefore, it was concluded that XPT was efficiently converting triplet excitons into emission through the singlet pathway by the TADF mechanism. The maximum EQE of XtBuCT device was 4% and was likely limited by its lower PLQY of 35%, which was almost half of XPT (66%).

In summary, three 9,9-dimethylxanthene bridged D-A molecules bearing phenothiazine, carbazole or 3,6-di-tert-butylcarbazole as donor groups were designed and synthesized. The rigid placement of a donor and an acceptor into a co-facial arrangement at a distance of greater than or equal to about 3.3 Angstroms and less than or equal to about 3.5 Angstroms produced quantitative formation of a charge transfer excimer structure. These “U-shaped molecules” exhibited delayed fluorescence in the absence of triplet-quenching oxygen in both solution and solid states, and hence, were characterized as TADF materials. These materials also showed enhanced quantum yields in the solid state and also displayed AIE behavior. The crystal structure analysis suggested C-H . . . pi interactions promoted a rigid environment that decreased non-radiative deactivation.

The OLED devices using XPT as the emitter displayed electroluminescence with a 10% EQE, which was higher than the theoretical limit of simple fluorescent OLEDs, thereby confirming that the devices were extracting light from both triplet to singlet excitons generated by charge recombination in the OLED devices. The molecular architectures reported provide a promising design for the development of further AIDF materials.

### Analysis Methods

High-resolution mass spectra (HRMS) were acquired by a Bruker Daltonics APEXIV 4.7 Tesla FT-ICR-MS employing electrospray (ESI) or direct analysis in real time (DART) as the ionization technique at the MIT Department of Chemistry Instrumentation Facility. Absorption spectra were measured with an Agilent Cary 4000 Series UV-Vis spectrometer. Fluorescence spectra were recorded with a Horiba Jobin Yvon SPEX Fluorolog-τ3 fluorimeter (model FL-321) with a 450 at xenon short-arc lamp. Thermogravimetric analysis (TGA) was performed on a TGAQ50 (TA instruments) under nitrogen atmosphere. The temperature was increased to 800° C. at 10° C./min. Differential scanning calorimetry (DSC) measurements were performed on a DSCQ10 calorimeter (TA instruments) under nitrogen atmosphere. The temperature was increased and decreased at 10° C./min. The measurements were repeated 3 times and 2^{nd} cycle was utilized to determine the glass transition temperature. Cyclic voltammetry was carried out with an AUTOLAB PGSTAT 10 potentiostat (Eco Chemie) on a three electrode system: a platinum working electrode (1.6 mm diameter), a platinum wire counter electrode and a quasi-internal silver wire submersed in 0.01 M AgNO₃/0.1M nBu₄NPF₆ in anhydrous acetonitrile as a reference electrode. The experiments were performed in freshly prepared solutions with 0.1 M nBu₄NPF₆ as an electrolyte in CH₂Cl₂ or THF distilled over CaH or sodium respectively with scan rate of 100 mV/s. The ferrocene/ferrocenium redox couple was used as an internal standard.

### DFT Calculations

The gas-phase ground state molecular geometry optimizations were performed by the ORCA^{1,2} software program with density functional theory (DFT) and in B3LYP hybrid exchange-correlation functional with the 6-31G* basic set were used. Using the optimized structures, time-dependent DFT (TD-DFT) calculations were carried out using the same functional and basis. The resolution of identity chain-of-spheres module, RIJCOSX¹, was used to reduce the computational cost of the calculations. The molecular orbitals were visualized using a Jmol software.

### Materials

Every solvent was ACS reagent grade or better, and used without further purification unless noted otherwise. Diethylether and tetrahydrofuran were refluxed and distilled over sodium under nitrogen atmosphere, which were kept over activated molecular sieves (4 Angstroms). Dichloromethane-d₂ and chloroform-d were purchased from Cambridge Isotope Laboratories, Inc. All reagent grade materials were purchased from commercial resources and used without further purification unless noted. Thin layer chromatography (Merck silica gel 60 F254 plates) was used for monitoring reaction progress. Silica Gel (60. pore size, 230-400 mesh) was used for purifying synthesized materials. 4,5-diiodo-9,9-dimethylxanthene was synthesized and purified by silica chromatography from hexane.

### Synthetic Procedures

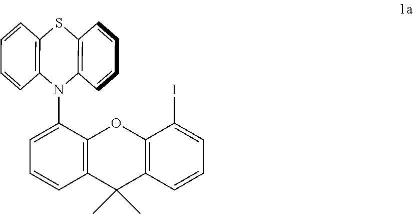

A mixture of 1.8 g of 4,5-diiodo-9,9-dimethyl-9H-xanthene (3.90 mmol), 0.813 g of phenothiazine (4.08 mmol), 4.41 g of potassium carbonate (31.9 mmol), 0.206 g of 18-Crown-6 (0.780 mmol) and 1.509 g of activated copper bronze (23.4 mmol) in 144 ml of 1,2-dichlorobenzene was stirred vigorously and refluxed under argon for 48 h. After cooling, chloroform was added to the solution, which was then filtered through filter paper (413, qualitative, medium flow) to get rid of cupper bronze. The filtrate was washed with saturated aqueous solution of NH₄Cl, water and brine. The organic layer was dried over MgSO₄, and the filtrated solution was evaporated under pressure to dryness. The crude product was purified with column chromatography on silica gel using gradient of 0-30% of dichloromethane in hexane. 1a was obtained as a colorless solid (1.23 g, 2.30 mmol, 59% yield).

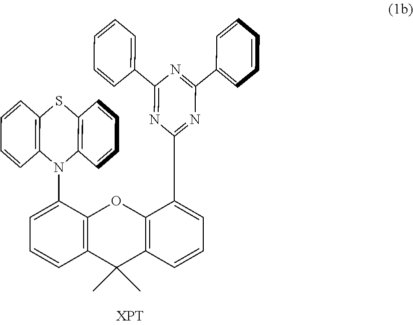

A dry and argon-flushed Schlenk-flask, equipped with a condenser and a magnetic stirring bar, was charged with 0.61 g of 2-iodo-4,6-diphenyl-1,3,5-triazine (1.70 mmol) in 10 ml of distilled THF, and cooled to −78° C. by methanol/dry ice bath. Then, 2 M n-butylMgCl in THF (0.93 ml, 1.86 mmol) was added with a syringe. The resulting orange solution was stirred for 10 minutes at the same temperature. After addition of 1 M ZnBr₂LiCl solution (1.86 ml, 1.86 mmol) at −78° C. and stirring for 15 minutes at the same temperature, the solution turned yellowish orange. A solution of 1a (0.825 g, 1.55 mmol), Pd₂(dba)₃ (70.96 mg, 0.0775 mmol), CPhos (101.5 mg, 0.2325 mmol) in distilled THF (10 ml) was added and followed by warming slowly to room temperature and refluxed for 16 h. The resulting dark red solution was diluted with chloroform. The organic layer was washed with 0.1 M HCl aqueous solution, water and brine. The organic layer was dried over MgSO₄, and the filtrated solution was evaporated under pressure to dryness. The crude product was purified with column chromatography on silica gel using gradient of 0-50% of dichloromethane in hexane. XPT was obtained as a yellow solid (0.535 g, 0.84 mmol, 54% yield).

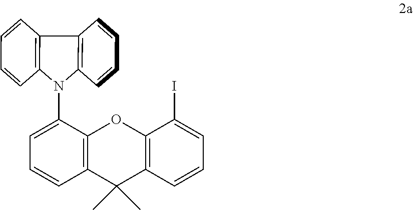

A mixture of 0.9 g of 4,5-diiodo-9,9-dimethyl-9H-xanthene (1.95 mmol), 0.342 g of carbazole (2.04 mmol), 2.21 g of potassium carbonate (15.99 mmol), 0.103 g of 18-Crown-6 (0.390 mmol) and 0.756 g of activated copper bronze (11.9 mmol) in 72 ml of 1,2-dichlorobenzene was stirred vigorously and refluxed under argon for 48 h. After cooling, chloroform was added to the solution, which was then filtered through filter paper (413, qualitative, medium flow) to get rid of cupper bronze. The filtrate was washed with saturated aqueous solution of NH₄Cl, water and brine. The organic layer was dried over MgSO₄, and the filtrated solution was evaporated under pressure to dryness. The crude product was purified with column chromatography on silica gel using gradient of 0-30% of dichloromethane in hexane. 2a was obtained as a colorless solid (0.55 g, 1.10 mmol, 56% yield).

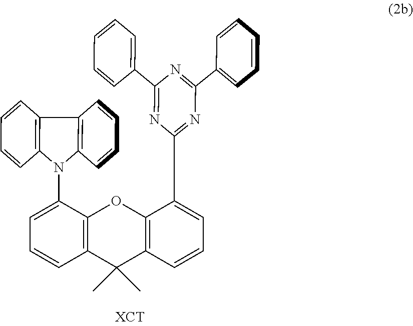

A dry and argon-flushed Schlenk-flask, equipped with a condenser and a magnetic stirring bar, was charged with 0.334 g of 2-iodo-4,6-diphenyl-1,3,5-triazine (0.93 mmol) in 5.1 ml of distilled THF, and cooled to −78° C. by methanol/dry ice bath. Then, 2 M n-butylMgCl in THF (0.51 ml, 1.02 mmol) was added with a syringe. The resulting orange solution was stirred for 10 minutes at the same temperature. After addition of 1 M ZnBr₂LiCl solution (1.02 ml, 1.02 mmol) at −78° C. and stirring for 15 minutes at the same temperature, the solution turned yellowish orange. A solution of 2a (0.424 g, 0.846 mmol), Pd₂(dba)₃ (38.7 mg, 0.0423 mmol), CPhos (55.4 mg, 0.127 mmol) in distilled THF (5.1 ml) was added and followed by warming slowly to room temperature and refluxed for 16 h. The resulting dark red solution was diluted with chloroform. The organic layer was washed with 0.1 M HCl aqueous solution, water and brine. The organic layer was dried over MgSO₄, and the filtrated solution was evaporated under pressure to dryness. The crude product was purified with column chromatography on silica gel using gradient of 0-50% of dichloromethane in hexane. XCT was obtained as a colorless solid (0.300 g, 0.494 mmol, 58% yield).

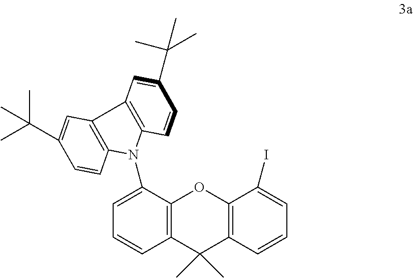

A mixture of 0.787 g of 4,5-diiodo-9,9-dimethyl-9H-xanthene (1.70 mmol), 0.50 g of 3,6-Di-tert-butylcarbazole (1.79 mmol), 1.93 g of potassium carbonate (13.94 mmol), 89.8 mg of 18-Crown-6 (0.340 mmol) and 0.659 g of activated copper bronze (10.37 mmol) in 63 ml of 1,2-dichlorobenzene was stirred vigorously and refluxed under argon for 48 h. After cooling, chloroform was added to the solution, which was then filtered through filter paper (413, qualitative, medium flow) to get rid of cupper bronze. The filtrate was washed with saturated aqueous solution of NH₄Cl, water and brine. The organic layer was dried over MgSO₄, and the filtrated solution was evaporated under pressure to dryness. The crude product was purified with column chromatography on silica gel using gradient of 0-20% of dichloromethane in hexane. 3a was obtained as a colorless solid (0.615 g, 1.00 mmol, 59% yield).

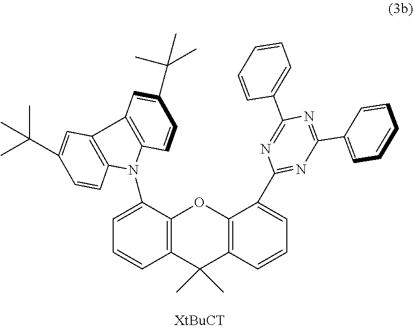

A dry and argon-flushed Schlenk-flask, equipped with a condenser and a magnetic stirring bar, was charged with 0.356 g of 2-iodo-4,6-diphenyl-1,3,5-triazine (0.99 mmol) in 6.0 ml of distilled THF, and cooled to −78° C. by methanol/dry ice bath. Then, 2 M n-butylMgCl in THF (0.54 ml, 1.08 mmol) was added with a syringe. The resulting orange solution was stirred for 10 minutes at the same temperature. After addition of 1 M ZnBr₂LiCl solution (1.08 ml, 1.08 mmol) at −78° C. and stirring for 15 minutes at the same temperature, the solution turned yellowish orange. A solution of 3a (0.552 g, 0.90 mmol), Pd₂(dba)₃ (41.0 mg, 0.045 mmol), CPhos (58.9 mg, 0.135 mmol) in distilled THF (6.0 ml) was added and followed by warming slowly to room temperature and refluxed for 16 h. The resulting dark red solution was diluted with chloroform. The organic layer was washed with 0.1 M HCl aqueous solution, water and brine. The organic layer was dried over MgSO₄, and the filtrated solution was evaporated under pressure to dryness. The crude product was purified with column chromatography on silica gel using gradient of 0-50% of dichloromethane in hexane. XtBuCT was obtained as a colorless solid (0.236 g, 0.328 mmol, 36% yield).

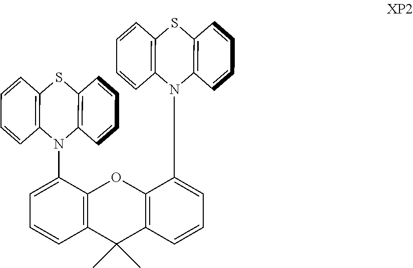

XP2 was obtained as the byproduct of the synthesis of al and isolated as a white solid.

### Photophysical Properties

The solutions of XPT, XCT and XtBuCT were prepared in 1.0×10⁴ M for UV-Vis measurements and 1.0×10⁻⁵ M for PL measurements. Thin films were spincoated on a glass substrate using the CHCl₃ solution of materials. Pictures were taken under a 365 nm LED-UV lamp.

### X-Ray Crystal Structures

Low-temperature diffraction measurements (Φ and ω-scans) were performed on a Siemens Platform three-circle diffractometer coupled to a Bruker-AXS X8 Kappa Duo diffractometer and a Smart Apex2 CCD detector with MO Kα radiation (λ=0.71073 Angstroms) from an IμS micro-source. The obtained structures and packing were visualized on a Mercury software. Table 4 summarizes the crystal data and structure refinement details for the three single crystals.

TABLE 4

| | XPT | | XCT | | XtBuCT | |
|---|---|---|---|---|---|---|
| Identification code | X16061 | | X16062 | | X16105 | |
| Empirical formula | C₄₂H₃₀N₄OS | | C₄₂H₃₀N₄O | | C₅₀H₄₆N₄O | |
| Formula weight | 638.76 | | 606.7 | | 718.91 | |
| Temperature (K) | 100 | (2) | 100 | (2) | 100 | (2) |
| Wavelength (Å) | 0.71073 | | 0.71073 | | 0.71073 | |
| Crystal system | Orthorhombic | | Orthorhombic | | Triclinic | |
| Space group | Pbca | | Pbca | | P-1 | |
| a (Å) | 20.1654 | (19) | 20.5502 | (7) | 8.3931 | (10) |
| b (Å) | 8.1152 | (8) | 7.9367 | (3) | 12.0824 | (14) |
| c (Å) | 37.841 | (4) | 37.7229 | (14) | 20.875 | (3) |
| α (°) | 90 | | 90 | | 75.674 | (3) |
| β (°) | 90 | | 90 | | 86.453 | (3) |
| γ (°) | 90 | | 90 | | 74.944 | (3) |
| Volume (Å³) | 6192.6 | (10) | 6152.6 | (4) | 1980.7 | (4) |

| | | | |
|---|---|---|---|
| Z | 8 | 8 | 2 |
| Density (calculated) (mg/m³) | 1.370 | 1.310 | 1.205 |
| Absorption coefficient (mm⁻¹) | 0.148 | 0.080 | 0.072 |
| F(000) | 2672 | 2544 | 764 |
| Crystal size (mm³) | 0.355 × 0.250 × 0.025 | 0.250 × 0.145 × 0.115 | 0.193 × 0.059 × 0.040 |
| 2θ for data collection (°) | 1.076 to 29.572 | 1.465 to 32.576 | 1.798 to 28.282 |
| Index ranges | −28 <= h <= 27, −11 <= | −30 <= h <= 29, −11 <= | −11 <= h <= 11, −16 <= |
| | k <= 11, −52 <= l <= 52 | k <= 12, −57 <= l <= 57 | k <= 16, −27 <= l <= 27 |
| Reflections collected | 170209 | 180118 | 175966 |
| Independent reflections | 8675 [R(int) = 0.0821] | 11167 [R(int) = 0.0448] | 9812 [R(int) = 0.0524] |
| Completeness to theta = 25.242° | 99.90% | 99.90% | 99.90% |
| Absorption correction | Semi-empirical | Semi-empirical | Semi-empirical |
| | from equivalents | from equivalents | from equivalents |
| Refinement method | Full-matrix least- | Full-matrix least- | Full-matrix least- |
| | squares on F² | squares on F2 | squares on F2 |
| Data/restraints/parameters | 8675/0/435 | 11167/0/426 | 9812/0/504 |
| Goodness-of-fit on F² | 1.091 | 1.052 | 1.035 |
| Final R indices [I > 2σ(I)] | R₁ = 0.0563, wR₂ = | R₁ = 0.0465, wR₂ = | R₁ = 0.0418, wR₂ = |
| | 0.1198 | 0.1227 | 0.1058 |
| R indices (all data) | R₁ = 0.0762, wR₂ = | R₁ = 0.0596, wR₂ = | R₁ = 0.0504, wR₂ = |
| | 0.1303 | 0.1327 | 0.1119 |
| Largest diff. peak and hole (e.Å⁻³) | 0.291 and −0.498 | 0.427 and −0.227 | 0.352 and −0.292 |

### Electrochemical Properties

The energy levels of the HOMO and LUMO were estimated from the cyclic voltammetry according to the formulas:

E_{HOMO}=−(E^{1/0}_{1/2}(vs. Fc⁺/Fc)+4.8)

E_{LUMO}=−(E^{0/-1}_{1/2}(vs. Fc⁺/Fc)+4.8)

except for irreversible peaks, where the formulas below were used:

E_{HOMO}=−(E^{1/0}ₒₙₛₑₜ(vs. Fc⁺/Fc)+4.8)

E_{LUMO}=−(E^{0/-1}ₒₙₛₑₜ(vs. Fc⁺/Fc)+4.8)

The optical bandgaps were determined from the onset of the absorption band (λₒₙₛₑₜ) in CH₂Cl₂. Optical bandgap E_{g}^{opt}=1240/λₒₙₛₑₜ

### Device Fabrication and Characterization

XPT and XCT were sublimated before use. ITO substrates were purchased from Thin Film Devices. The substrates were cleaned with acetone and isopropyl alcohol in an ultrasonic bath followed by boiling in isopropyl alcohol for 5 minutes. Then the ITO substrates were treated with UV-ozone plasma to clean the surface and to increase the work function of ITO. All organic layers and cathode were deposited by a vacuum thermal evaporator with base pressure below 5×10⁻⁷ torr. The devices were encapsulated with a cover glass using UV curable epoxy in a glovebox where both oxygen and moisture levels were kept below 1 ppm. The current-voltage-luminescence characteristics of the devices were measured by HP-4156C parameter analyzer linked to a NIST-calibrated silicon photodetector, FDS1010-CAL. The EL spectrum was obtained with a spectrometer (SP-2300, Princeton Instruments).

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A device, comprising:
an electrode; and
an emissive compound in electrical communication with the electrode, the emissive compound comprising a tricyclic aromatic group, optionally substituted,
wherein the emissive compound comprises a donor and an acceptor, each bound to the tricyclic aromatic group,
wherein the donor comprises an optionally substituted phenothiazine group or an optionally substituted carbazole group, and
wherein the acceptor comprises an optionally substituted diphenyltriazine group.

2. A device as in claim 1, wherein an intramolecular distance between the donor and acceptor is less than or equal to 5 angstroms.

3. A device as in claim 1, wherein the emissive compound has a structure as in Formula (I): 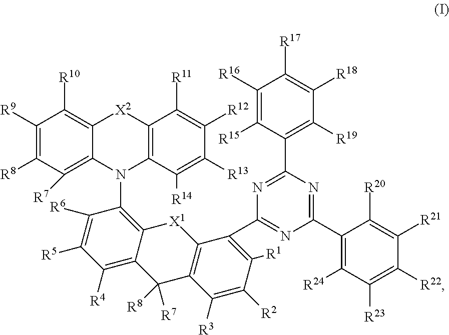
wherein:
X¹ is S, O, or absent,
X² is S, O, N, CH—R²⁵, or absent, and
R¹-R²⁵ are the same or different and are each hydrogen, halo, hydroxyl, amino, alkyl, heteroalkyl, alkenyl, heteroalkenyl, alkynyl, aryl, heteroayrl, heterocyclyl, or carbonyl group, any of which is optionally substituted, and/or any two adjacent groups of R¹-R²⁵ can be joined together to form an optionally substituted ring.

4. A device as in claim 1, wherein X¹ is oxygen, X² is sulfur or absent, R⁷ and R⁸ are each hydrogen or methyl, R⁹ and R¹² are each hydrogen or tert-butyl, and R¹-R⁶, R¹⁰-R¹¹, R¹³-R²⁵ are each hydrogen.

5. A device as in claim 1, wherein the emissive compound has a structure as in Formula (II), Formula (III), or Formula (IV):

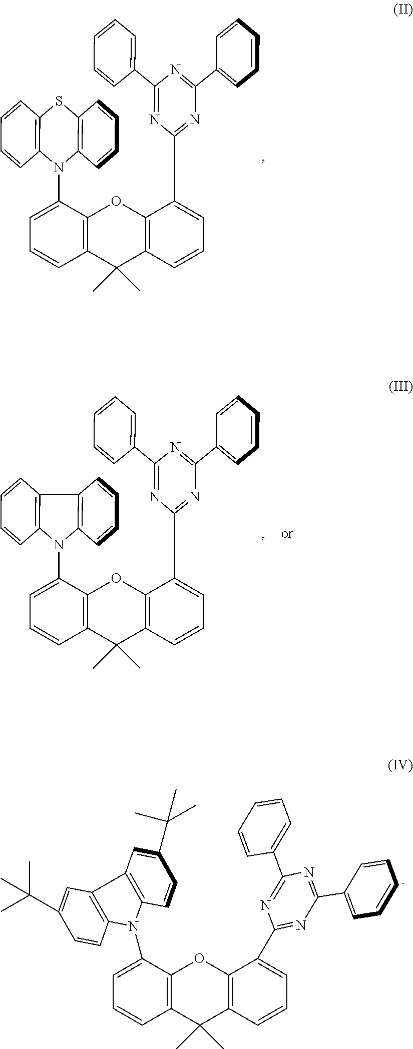

6. A device as in claim 1, wherein an excited state lifetime of the emissive compound is greater than or equal to 30 nanoseconds.

7. A device as in claim 1, wherein an efficiency of an emission of the emissive compound is increased in the solid state relative to solution.

8. A device as in claim 1, wherein a wavelength of an emission of the emissive compound is less than 500 nm.

9. A device as in claim 1, wherein an emission of the emissive compound is characterized as giving blue light.

10. A device as in claim 1, wherein the emissive compound emits blue light when excited in a solid state.

11. A composition comprising:
an emissive compound comprising a tricyclic aromatic group, optionally substituted, wherein the emissive compound comprises a donor group and an acceptor group, each bound to the tricyclic aromatic group,
wherein the donor and acceptor are co-facially aligned, and
wherein the highest occupied molecular orbital is localized on the donor and the lowest unoccupied molecular orbital is localized on the acceptor.

12. A composition as in claim 11, wherein an intramolecular distance between the donor and acceptor is less than or equal to 5 angstroms.

13. A composition as in claim 11, wherein the donor comprises an optionally substituted phenothiazine group or an optionally substituted carbazole group.

14. A composition as in claim 11, wherein the acceptor comprises an optionally substituted diphenyltriazine group.

15. A composition comprising:
an emissive compound having a structure as in Formula (I): 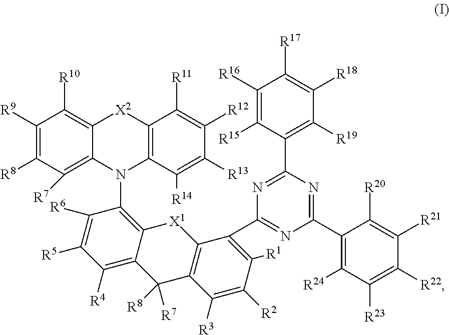
wherein:
X¹ is S, O, or absent,
X² is S, O, N, CH—R²⁵, or absent, and
R¹-R²⁵ are the same or different and are each hydrogen, halo, hydroxyl, amino, alkyl, heteroalkyl, alkenyl, heteroalkenyl, alkynyl, aryl, heteroayrl, heterocyclyl, or carbonyl group, any of which is optionally substituted, and/or any two adjacent groups of R¹-R²⁵ can be joined together to form an optionally substituted ring.

16. A composition as in claim 15, wherein X¹ is oxygen, X² is sulfur or absent, R⁷ and R⁸ are each hydrogen or methyl, R⁹ and R¹² are each hydrogen or tert-butyl, and R¹-R⁶, R¹⁰-R¹¹, R¹³-R²⁵ are each hydrogen.

17. A composition as in claim 15, wherein the emissive compound has a structure as in Formula (II), Formula (III), or Formula (IV):

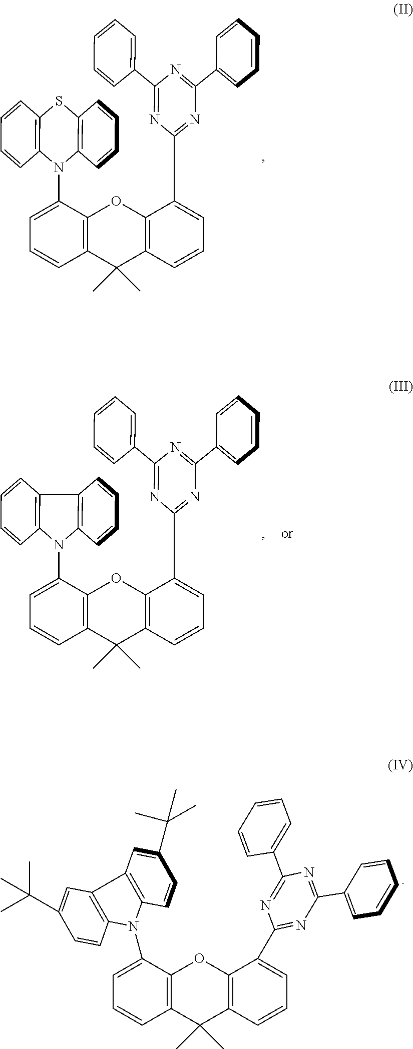

18. A composition as in claim 15, wherein an excited state lifetime of the emissive compound is greater than or equal to 30 nanoseconds.

19. A composition as in claim 15, wherein an efficiency of an emission of the emissive compound is increased in the solid state relative to solution.

20. A composition as in claim 15, wherein a wavelength of an emission of the emissive compound is less than 500 nm.

21. A composition as in claim 15, wherein an emission of the emissive compound is characterized as giving blue light.

22. A composition as in claim 15, wherein the emissive compound emits blue light when excited in a solid state.

23. A composition as in claim 15, wherein the composition has a quantum yield of greater than or equal to 35% in the solid state.

24. A composition as in claim 15, wherein the composition has a thermally activated delayed fluorescence lifetime between 1 microsecond and 10 microseconds.

25. An organic light emitting device, comprising:
a composition as in claim 15; and
two electrodes constructed and arranged to be in electrical communication with the composition.

26. A organic light emitting device as in claim 25, wherein the organic light emitting device has a maximum electroluminescence external quantum efficiency of greater than or equal to 4% and less than or equal to 11%.
